(19) United States
(12) Patent Application Publication
(10) Pub. No.: US 2025/0249198 A1
(43) Pub. Date: Aug. 7, 2025

(54) System for the Decoupled Supply and Conservation of Oxygen and other Substances

(71) Applicant: OXFO Corporation, Chestnut Hill, MA (US)

(72) Inventors: Carlos Fernando Bazoberry, Chestnut Hill, MA (US); Brent H. Young, Boston, MA (US)

(21) Appl. No.: 19/044,621

(22) Filed: Feb. 3, 2025

Related U.S. Application Data
(63) Continuation-in-part of application No. 17/068,718, filed on Oct. 12, 2020, now Pat. No. 12,214,126.
Application No. 17/068,718 is a continuation-in-part of application No. 16/358,666, filed on Mar. 19, 2019, now Pat. No. 10,800,589.
(60) Provisional application No. 63/056,944, filed on Jul. 27, 2020.

(51) Int. Cl. A61M 16/10; A61M 16/20

(52) U.S. Cl. CPC A61M 16/1015; A61M 16/208; A61M 2205/0272; A61M 2205/3327; A61M 2205/3344; A61M 2205/3389

(57) ABSTRACT

A system and method for conserving oxygen and other gases supplied to a recipient. A supply conduit supplies gas from a source to a reservoir of lightweight, flexible film retaining a volume of gas at ambient pressure. A conduit supplies gas from the reservoir to the recipient. An inflation detection system, which may include plural distance sensors, detects when the reservoir is below a state of minimum inflation and when the reservoir is inflated to a maximum state of inflation. A valve system prevents gas from flowing from the source and into the reservoir when the reservoir reaches the predetermined maximum state of inflation, and the valve system permits gas to flow from the source into the reservoir when the reservoir reaches the predetermined minimum state of inflation whereby gas within the reservoir can be continually replenished without pressurization above ambient pressure.

### RELATED APPLICATIONS

This application is a Continuation-in-Part of application Ser. No. 17/068,718, filed Oct. 12, 2020, which claimed priority to Provisional Patent Application No. 63/056,944, filed Jul. 27, 2020, and which was a Continuation-in-Part of application Ser. No. 16/358,666, filed Mar. 19, 2019. The entirety of each of the foregoing is incorporated herein by reference.

### FIELD OF THE INVENTION

The present invention relates generally to the delivery of gases from a source to a recipient wherein an ambient-pressure decoupling reservoir enables a decoupling of the process of demand from the reservoir to a recipient from the process of supply to the reservoir from the source. More particularly, disclosed herein are a system and method for conserving oxygen and other gases and substances when delivering them from the decoupling reservoir to a recipient at ambient pressure with automatic refilling of the decoupling reservoir from a source. In embodiments of the system and method, oxygen and other gases and substances are transferred from the decoupling reservoir to a recipient on demand by the recipient via a pressure differential between them, and the decoupling reservoir is automatically refilled with gas from the source.

### BACKGROUND OF THE INVENTION

The supply of oxygen can be a critical need for hospital patients and others. Meanwhile, in developing countries and during times of increased demand in all places, shortages of oxygen and excessive costs can place extreme limits on availability and can jeopardize the health and safety of patients in need. For instance, during the COVID-19 pandemic, the demand for oxygen left hospitals and other caregiving institutions in dire need of the life-saving gas. One headline from the AP News network on Jun. 24, 2020 warned, “Scarce Medical Oxygen Worldwide Leaves Many Gasping for Life.” One day later, Reuters observed, “WHO Warns of Oxygen Shortage as COVID Cases Set to Top 10 Mln” with the World Health Organization estimating based on there being approximately one million new coronavirus cases worldwide per week that the world would need 620,000 cubic meters of oxygen per day, which roughly equals 88,000 large cylinders, for COVID-19 patients alone.

According to the prior art, one way that supplemental oxygen is supplied to patients is via a fluidic connection, typically extension tubing, between a pressurized source of oxygen, such as an oxygen cylinder or tank, and the patient. The pressurized source supplies a constant flow of oxygen to the patient travelling from the tank continuously through the connector tubing, regardless of whether the patient is breathing in or out. Even while the patient exhales and thus cannot intake oxygen, the oxygen constantly flows and is wasted. Indeed, half or even more of the constantly supplied oxygen in such continuous pressure feed methods is wasted and expelled to the atmosphere.

In times of great need, it is apparent that conserving oxygen resources with one patient may well save the life of another. Moreover, conserving oxygen not only reduces the overall need per patient but also tends to reduce the cost of oxygen per unit. Meanwhile, it is clear that each patient requires a sufficient supply of oxygen. The challenge is thus how to provide oxygen in sufficient supply on demand while minimizing waste.

To comprehend a solution to that challenge, one must understand what drives the flow of air containing oxygen into the lungs, how flow is normally initiated and maintained between the patient and ambient air, and how alveolar pressure varies while pleural pressure decreases throughout inspiration. Air, like other fluids, moves from a region of higher pressure to a region of lower pressure. The flow of air into the lungs requires the establishment of a pressure gradient between the atmosphere and the alveoli. This driving pressure gradient is accomplished by the contraction of the inspiratory muscles. Contraction of the inspiratory muscles expands the chest wall, lowering the pressures in the thoracic cavity so that intra-pleural and alveolar pressure decrease according to Boyle's law. Muscle contraction results in a change in thoracic volume, leading to a change in alveolar pressure, which in turn provides the driving pressure for air flow into the lungs.

Normally, the lungs absorb oxygen from the air during breathing. However, certain conditions can prevent a person from getting enough oxygen. As a result, oxygen therapy with oxygen delivery equipment is required. Patients can receive oxygen therapy from a source of oxygen through tubes resting in their nose, through a facemask, or through a tube placed in their trachea or windpipe. Oxygen treatment increases the amount of oxygen the lungs receive and deliver to the blood. Oxygen therapy may be prescribed for a patient when the patient has a condition that causes the patient's blood oxygen levels to be too low. Low blood oxygen may make patients feel short of breath, tired, or confused and can damage the patient's body. Oxygen therapy may be needed on a temporary basis, such as due to a treatable respiratory illness, or on a long-term basis. Often, the source of oxygen is a tank of compressed oxygen gas or liquid.

Oxygen tanks must be produced, transported, stored, and refilled on a continual basis. In exigent circumstances, such as during an epidemic or a pandemic involving respiratory distress, need can dangerously outpace supply. Moreover, in remote and economically challenged locations, providing ample replenishment of oxygen supplies can be highly costly, even catastrophically impossible. Meanwhile, with life-saving oxygen often in preciously short supply and with oxygen constantly passed through tubing to the patient, at least half of the supplied oxygen is simply exhausted into the atmosphere, including during exhalation during which the entirety of the supplied oxygen is wasted.

In view of the foregoing, the present inventors appreciated the critical need for a system and method capable of providing a ready supply of oxygen to a patient while minimizing or eliminating wasted oxygen thereby minimizing the supply needs of individual patients, maximizing the effective supply of oxygen, and enabling better patient care and optimal health outcomes in a cost-efficient manner even in times of public health crises.

### SUMMARY OF THE INVENTION

Knowing the critical need for ample supplies of oxygen to patients, a primary object of the present invention is to save lives.

The present inventors further set forth with a basic object of providing a system and method for supplying oxygen and other flowable substances to patients and other recipients in need that reduces wasted oxygen thereby to maximize the effective use of available oxygen supplies.

A further object of embodiments of the invention is to provide a system and method for supplying oxygen and other flowable substances to recipients that enable an ample supply of oxygen on-demand while minimizing or eliminating inefficient oxygen losses.

Another object of manifestations of the invention is to provide a system and method for supplying oxygen and other flowable substances to recipients that maximize efficiencies in usage and minimize supply costs.

These and further objects, advantages, and details of the present invention will become obvious not only to one who reviews the present specification and drawings but also to those who have an opportunity to observe the systems and methods disclosed herein in operation. However, it will be appreciated that, although the accomplishment of plural of the foregoing objects in a single embodiment of the invention may be possible and indeed preferred, not all embodiments will seek or need to accomplish each and every potential advantage and function. Nonetheless, all such embodiments should be considered within the scope of the present invention.

One will appreciate that the foregoing discussion broadly outlines the more important goals and features of the invention to enable a better understanding of the detailed description that follows and to instill a better appreciation of the inventors' contribution to the art. Before any particular embodiment or aspect thereof is explained in detail, it must be made clear that the following details of construction and illustrations of inventive concepts are mere examples of the many possible manifestations of the invention.

In carrying forth one or more of the foregoing objects, an embodiment of the present invention can be characterized as a system for the conservation of oxygen supplied to a patient. The system has an expandable and compressible decoupling donor reservoir that has an outer wall, an inner volume for retaining a volume of oxygen, and at least one orifice for allowing a passage of oxygen into and out of the inner volume. As disclosed herein, the decoupling reservoir comprises a shell of flexible material that acts as an envelope for retaining a volume of oxygen at ambient pressure throughout the entire breathing cycle simply waiting to be naturally inhaled by the patient.

Based on its structural properties as taught herein, including the material, material thickness, and structural configuration of the shell, the shell of the decoupling reservoir and the decoupling reservoir itself provide no or substantially no resistance to inflation when receiving oxygen from a source, and the decoupling reservoir provides no or substantially no resistance to deflation when oxygen is inhaled therefrom by a patent. When maintained at ambient pressure as taught herein, the material of the shell of the decoupling reservoir and the decoupling reservoir itself are incapable of acquiring or exhibiting elastic potential energy. As such, within the bounds of ambient pressure, a decoupling reservoir that is deflated will not tend to resiliently spring and expand toward an expanded configuration, and a decoupling reservoir that is inflated will not tend to resiliently spring and contract toward a retracted configuration. As disclosed herein, the entire shell of the decoupling reservoir can be formed of such a material that is incapable of acquiring or exhibiting elastic potential energy at ambient pressure. The decoupling reservoir could have one or more rigid shell portions joined with one or more flexible shell portions formed of such a material that is incapable of acquiring or exhibiting elastic potential energy.

One non-limiting material for the decoupling reservoir that is incapable of acquiring or exhibiting elastic potential energy at ambient pressure is a lightweight, flexible foil. One such flexible foil is founded on a film of polymeric material, such as a film of polyester and even more particularly a film of biaxially oriented polyethylene terephthalate (BOPET). The film of polymeric material can be metalized, such as with an aluminum film lining. In one non-limiting practice, a film of aluminum or potentially another metal can be evaporated onto the film of polymeric material thereby further reducing the permeability of the foil. For avoidance of doubt, the term “foil” as used herein shall not be interpreted to require metallization except as expressly set forth.

A supply conduit is adapted to receive oxygen from a source of oxygen and to supply the same to the decoupling donor reservoir. The supply conduit has a first end for supplying oxygen to the donor reservoir and a second end for being fluidically connected to the source of oxygen, and an ambient pressure conduit is adapted to supply oxygen along a fluid path from the decoupling reservoir to a recipient. The ambient pressure conduit has a first end in fluidic communication with the decoupling reservoir, potentially through a connector, for receiving oxygen from the decoupling reservoir and a second end for being fluidically connected to the recipient.

An inflation detection system is operable to detect a first condition wherein the decoupling reservoir is inflated with oxygen to a predetermined state of inflation and a second condition wherein the decoupling reservoir is below the predetermined state of inflation. Finally, a valve system is disposed between the source of oxygen and the decoupling reservoir. The valve system is operative in a closed condition to prevent oxygen from flowing from the source of oxygen and into the decoupling reservoir when the decoupling reservoir is in the first condition, and the valve system is operative in an open condition to permit oxygen to flow from the source of oxygen and into the decoupling reservoir when the decoupling reservoir is in the second condition. Under this construction, oxygen can be made available within the envelope defined by the decoupling reservoir for natural inhalation by a patient, such as through a patient breathing mask as the recipient, and the decoupling reservoir can be automatically replenished to the predetermined state of inflation in decoupled inhalation and inflation processes.

In practices of the system, the valve system and the inflation detection system are operative to maintain the volume of oxygen in the decoupling reservoir substantially at ambient pressure. For instance, the decoupling reservoir can be considered to have a fully inflated condition, and the inflation detection system can be operative to detect when the decoupling reservoir is inflated to an upper end of a predetermined range of the fully inflated condition. The inflation detection system can then detect the first condition when the decoupling reservoir is inflated to the upper end of the predetermined range of the fully inflated condition, and the inflation detection system can detect the second condition when the decoupling reservoir is inflated or, more particularly, collapsed below a lower end of the predetermined range of the fully inflated condition.

In certain embodiments, the inflation detection system comprises an electro-mechanical system. For instance, the inflation detection system can comprise a switch disposed to be moved by the outer wall of the decoupling reservoir when the decoupling reservoir is inflated with oxygen to the predetermined state of inflation, such as the upper end of the range of the fully inflated condition. The switch can be biased, such as by gravity, by a resiliently compressible member, or by any other effective method, toward the decoupling reservoir. The switch can be considered to have an activated state wherein the switch is disposed at or beyond an inward position with respect to the inner volume of the decoupling reservoir and a deactivated state when the switch is moved outwardly by the outer wall of the decoupling reservoir when the volume of oxygen in the decoupling reservoir reaches the predetermined state of inflation. The valve system is operative to prevent oxygen from flowing from the source of oxygen and into the decoupling reservoir when the switch is in the deactivated state, and the valve system is operative to permit oxygen to flow from the source of oxygen and into the decoupling reservoir when the switch is in the activated state.

In particular manifestations of the system, the switch comprises a float switch. For example, the float switch can have a contact structure with a collar that is extendable and retractable relative to a central column. The collar can then retain a magnet, and the central column can then retain electrical contacts that are brought into electrical contact by a proximity of the magnet when the switch is in the activated state.

According to practices of the system, the valve system can take the form of a solenoid valve that is in electrical communication with the inflation detection system. The solenoid valve can be induced by the inflation detection system to a closed condition to prevent the flow of oxygen from the source of oxygen to the decoupling donor reservoir when the decoupling donor reservoir is in the first condition, and the solenoid valve can be induced by the inflation detection system to an open condition to permit the flow of oxygen from the source of oxygen to the decoupling donor reservoir when the decoupling donor reservoir is in the second condition.

A recipient delivery device, such as a patient breathing mask or another recipient delivery device, can be coupled to the second end of the ambient pressure conduit. Further, in certain embodiments, the decoupling reservoir can be disposed within a housing, which could comprise a main housing of the system, a sub-housing within a main housing, or some other type of housing. In other practices, the decoupling reservoir can be disposed without a housing. Where a housing is provided, the inflation detection system can, for instance, comprise an electro-mechanical system with a switch supported by the housing and disposed to be moved by the outer wall of the decoupling reservoir when the decoupling reservoir is inflated with oxygen to the predetermined state of inflation. Even more particularly, the housing can be transparent such that the state of inflation of the decoupling reservoir can be visually perceived.

Embodiments of the system can further incorporate a one-way inspiratory valve disposed along the fluid path from the decoupling reservoir to the recipient. The one-way inspiratory valve can be operative to enable oxygen to flow from the decoupling reservoir, through the ambient pressure conduit, and to the recipient but to prevent reverse flow of oxygen.

In alternative practices of the invention, the inflation detection system comprises a contactless detection system. For instance, the inflation detection system can take the form of an optical detection system operative to detect the state of inflation of the decoupling reservoir, such as the distance of one surface of the decoupling reservoir from a fixed location. In such practices, the opposing surface of the decoupling reservoir will preferably be fixed in place, such as by being adhered or otherwise fixed within the housing, to ensure an accurate determination of the state of inflation of the decoupling reservoir.

While the present invention is largely described as being employed to supply oxygen to human or other living patients in a manner that conserves oxygen supply, it will be understood that the invention is not limited to retaining and dispensing oxygen. Indeed, other gases and mixtures of gases and other fluids are possible within the scope of the invention. To that extent, embodiments of the invention can be more broadly characterized as a system for providing a supply of gas. Moreover, the gas need not necessarily be supplied to a patient. Other recipients are contemplated and within the scope of the invention except as may be expressly excluded by the claims.

Embodiments of the system can be characterized as a system for the decoupled supply and conservation of gas to a patient. The system is founded on an expandable and collapsible decoupling reservoir with an outer wall defining a shell, an inner volume for retaining a volume of gas, and at least one orifice for allowing a passage of gas into and out of the inner volume. The outer wall of the decoupling reservoir comprises, potentially consists of, a lightweight, flexible film. Alternatively described, the outer wall of the decoupling reservoir is formed from a material incapable of acquiring or exhibiting elastic potential energy when the decoupling reservoir is at ambient pressure. With that, the decoupling reservoir when deflated will not tend to resiliently spring and expand toward an expanded configuration, and the decoupling reservoir when inflated will not tend to resiliently spring and contract toward a collapsed configuration. A supply conduit is adapted to receive gas from a source of gas, and an ambient pressure conduit is adapted to supply gas along a fluid path from the decoupling reservoir to a recipient. An inflation detection system is operable to detect a first condition wherein the decoupling reservoir is inflated with gas to a predetermined maximum state of inflation and a second condition wherein the decoupling reservoir is deflated to a predetermined minimum state of inflation, and a valve system is disposed between the source of gas and the decoupling reservoir. The valve system is operative when in a closed condition to prevent gas from flowing from the source of gas and into the decoupling reservoir when the decoupling reservoir is in the first condition, and the valve system is operative in an open condition to permit gas to flow from the source of gas and into the decoupling reservoir when the decoupling reservoir is in the second condition. The valve system and the inflation detection system are operative to maintain the volume of gas in the decoupling reservoir substantially at ambient pressure. Where the decoupling reservoir is considered to have a fully inflated condition, the predetermined maximum state of inflation can be less than the fully inflated condition. Moreover, the decoupling reservoir has a fully deflated condition with the predetermined minimum state of inflation being less than the predetermined maximum state of inflation but greater than the fully deflated condition.

The decoupling reservoir can be disposed within a housing, and the inflation detection system can comprise a distance sensor retained by the housing that is operative to detect a distance of the outer wall of the decoupling reservoir from the distance sensor. In such embodiments, the inflation detection system is operative to detect the first condition and the second condition based on the distance of the outer wall of the decoupling reservoir from the distance sensor. Moreover, plural distance sensors retained by the housing can cooperate to detect a contour of the decoupling reservoir based on localized distances of the outer wall of the decoupling reservoir from respective distance sensors and, based on the contour of the decoupling reservoir and the localized distances of the decoupling reservoir from respective distance sensors, the state of inflation of the decoupling reservoir.

In practices of the system, the outer wall of the decoupling reservoir comprises or rather consists of a lightweight, flexible film of polymeric material. For instance, the outer wall of the decoupling reservoir can comprise a polyester film, which can be metalized, such as by a coating of aluminum. In such embodiments, the outer wall of the decoupling reservoir can, for example, be formed from a film of biaxially oriented polyethylene terephthalate. In other embodiments, the outer wall of the decoupling reservoir can be formed from a lightweight, flexible film of polyethylene, such as low density polyethylene. The lightweight, flexible film comprising the outer wall of the decoupling reservoir will preferably have a thickness of less than 100 microns (0.004 inches). Even more preferably, the lightweight, flexible film comprising the outer wall of the decoupling reservoir will have a thickness of less than 25 microns (0.001 inches). Still more particularly, where the lightweight, flexible film comprising the outer wall of the decoupling reservoir comprises a film of biaxially oriented polyethylene terephthalate, the film will ideally have a thickness between 2 and 20 microns (between 0.0000787 inches and 0.000787 inches) to achieve the performance characteristics sought pursuant to the invention. Where the lightweight, flexible film comprising the outer wall of the decoupling reservoir comprises a film of low density polyethylene, it will most ideally have a thickness between 5 and 20 microns (between 0.000197 inches and 0.000787 inches).

As disclosed herein, the lightweight, flexible film can, in certain embodiments, span and define the entire outer wall of the decoupled reservoir. However, it is also within the scope of the invention for the lightweight, flexible film to span only a portion of the outer wall of the decoupled reservoir while a rigid or semi-rigid material defines a portion of the outer wall of the reservoir. For example, the lightweight, flexible film of the decoupling reservoir can form a concave bowl when inflated while a rigid shell portion forming a concave bowl is joined with the concave bowl defined by the flexible film to form the decoupling reservoir. The concave bowls can be disposed in any effective orientation, including but not limited to by having the concave bowl formed by the rigid shell portion in an upturned position thus defining a lower portion of the decoupled reservoir and by having the concave bowl formed by the flexible material in a downturned position defining an upper portion of the decoupled reservoir.

One will appreciate that the foregoing discussion broadly outlines the more important goals and features of the invention to enable a better understanding of the detailed description that follows and to instill a better appreciation of the inventors' contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

In the accompanying drawing figures:

FIG. 1 is a schematic view of a system for the decoupled supply and conservation of oxygen and other substances, alternatively referred to as an automatic system for the conservation of gas, according to the present invention;

FIG. 2 is a schematic view depicting a series of respiratory cycles employing the system disclosed herein;

FIG. 3 is a top plan view of an alternative embodiment of the system for the decoupled supply and conservation of oxygen and other substances;

FIG. 4 is a view in front elevation of the system of FIG. 3;

FIG. 5 is a lateral perspective view of the system of FIG. 3;

FIG. 6 is an upper perspective view of an inflation detection system for the system for the decoupled supply and conservation of oxygen and other substances in an ON condition;

FIG. 7 is a lower perspective view of the inflation detection system again in an ON condition;

FIG. 8 is a view in side elevation of the inflation detection system in an OFF condition;

FIG. 9 is a top plan view of a system for the decoupled supply and conservation of oxygen and other substances as disclosed herein with the cover portion and the retained inflation detection system removed;

FIG. 10 is a bottom plan view of the system of FIG. 9;

FIG. 11 is a top plan view of an alternative system for the decoupled supply and conservation of oxygen and other substances according to the invention;

FIG. 12 is an amplified top plan view of the system of FIG. 11;

FIG. 13 is a bottom plan view of the system of FIG. 11;

FIG. 14 is a view in side elevation of the system of FIG. 11;

FIG. 15 is an anterior perspective view of the system of FIG. 11;

FIG. 16 is a perspective view of a filter and one-way inspiratory valve for the system for the decoupled supply and conservation of oxygen and other substances of FIG. 11;

FIG. 17 comprises schematic top plan and side elevation views of another system for the decoupled supply and conservation of oxygen and other substances according to the invention;

FIG. 18 comprises schematic top plan and side elevation views of still another system for the decoupled supply and conservation of oxygen and other substances according to the invention;

FIG. 19 is a view in side elevation of another embodiment of the system for the decoupled supply and conservation of oxygen and other substances according to the invention with the decoupling reservoir in an inflated condition;

FIG. 20 is a view in side elevation of the system of FIG. 19 with the decoupling reservoir in a partially deflated condition;

FIG. 21 is a sectioned top plan view of the system of FIG. 19;

FIG. 22 is an exploded perspective view of a fluidic connector for the system for the decoupled supply and conservation of oxygen and other substances;

FIG. 23 is a perspective view of an alternative decoupling reservoir according to the present invention; and

FIG. 24 is a perspective view of a disposition of the rigid shell portion of the decoupling reservoir disposed within a lower portion of a casing for a system as taught herein.

### DETAILED DESCRIPTION OF PREFERRED EMBODIMENTS

The system for the decoupled supply and conservation of oxygen and other substances disclosed herein are subject to a wide variety of embodiments. However, to ensure that one skilled in the art will be able to understand and, in appropriate cases, practice the invention disclosed herein, certain preferred embodiments of the broader invention are described below and shown in the accompanying drawing figures.

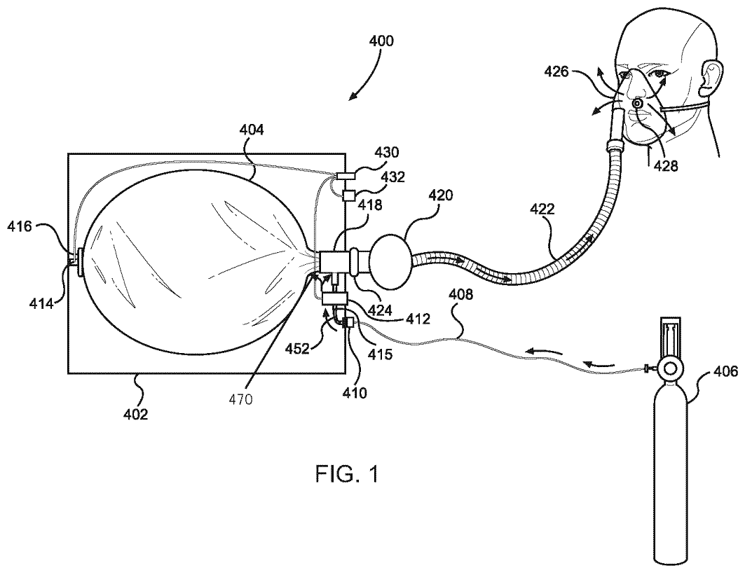

Looking more particularly to the drawings, the structure and operation of a system for the decoupled supply and conservation of oxygen and other substances 400, alternatively referred to as an automatic gas conservation system 400, according to the present invention can be understood with reference to FIG. 1. As shown and described herein, the system 400 provides an on-demand supply of oxygen at ambient pressure to a recipient, such as to a patient through a breathing mask 426, from a donor reservoir 404, which may alternatively be referred to as a decoupling reservoir 404 or a decoupling donor reservoir 404. The decoupling reservoir 404 retains oxygen at ambient pressure and is continually supplied with oxygen from an oxygen source 406, such as a tank of compressed oxygen gas or liquid oxygen.

With the decoupling reservoir 404 retaining oxygen at ambient pressure, a full and ample supply of oxygen is constantly available for patient inspiration. Concomitantly, oxygen losses during patient expiration are substantially eliminated thereby conserving the supply of oxygen without compromising availability to the individual recipient since oxygen within the decoupling reservoir 404 is automatically replenished in a process decoupled from the process of inhalation of oxygen from the decoupling reservoir 404 by the patient. While oxygen delivery is directly tied to a prescribed flow rate in traditional continuous flow system such that varying demand is not accommodated, the decoupling reservoir 404 of the disclosed system 400 acts as a buffer, providing an enveloped volume of oxygen waiting to be drawn in by the patient's lungs on demand. Sufficient and consistent oxygen supplies and concentrations are achieved without regard to changes in inspiratory flow volumes and rates.

The decoupling reservoir 404 in this embodiment comprises an expandable and compressible shell, bladder, or other expandable and compressible body that is disposed within a housing 402, which could be a primary housing or a sub-housing within a larger structure. However, the donor reservoir 404 need not necessarily be within a housing 402 to be within the scope of the invention. The housing 402 defines boundaries for the reservoir 404 so that the shell of the reservoir 404 presses toward one or more portions of the boundary defined by the housing 402 as the reservoir 404 is expanded. In this non-limiting example, the housing 402 has a bottom that defines a lower boundary for the reservoir 404, a top that defines an upper boundary for the reservoir 404, and distal ends that define longitudinal boundaries for the reservoir 404. Here, the reservoir 404 has an oblong, egg shape, and the housing 402 has a general cube shape, but other shapes and combinations of shapes are readily possible and within the scope of the invention except as it might be expressly limited by the claims.

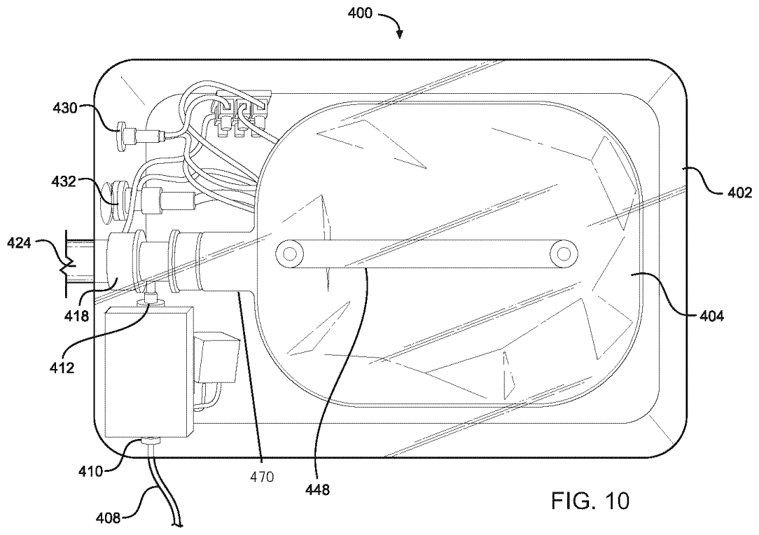

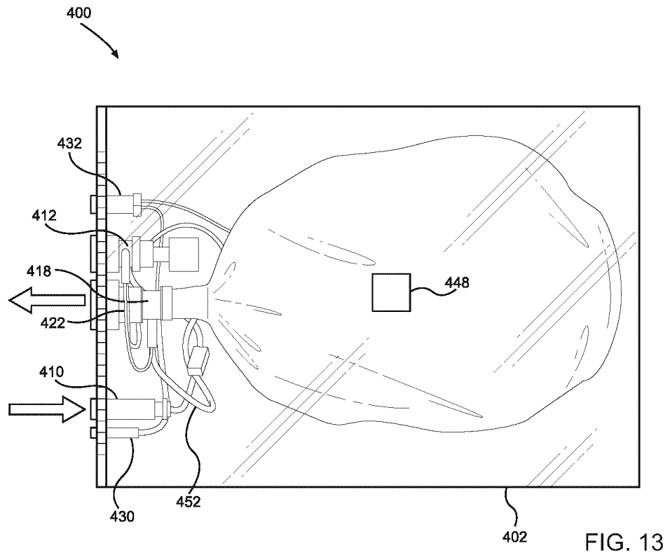

As in the embodiments of the system 400 shown in FIGS. 10 and 13, for example, the lower wall portion of the shell of the reservoir 404 can be adhered or otherwise secured to the bottom of the housing 402, such as by an adhesive strip 448 or in any other manner. With the lower wall portion of the shell of the reservoir 404 so secured, the lower wall portion of the shell of the reservoir 404 remains in a fixed, known position while the opposing upper wall portion of the shell of the reservoir 404 is permitted to expand and collapse freely with the flow of oxygen into the reservoir 404 from the source 406 and the flow of oxygen out of the reservoir 404 on inhalation by the patient. The volume and state of inflation of the reservoir 404 can be determined based on a sensing of, for example, the position of the opposing upper wall portion of the shell of the reservoir 404, whether by a contactless distance sensor, a contact switch, or any other inflation detection system. Ideally, a substantial portion of the lower wall portion of the shell of the reservoir 404, such as approximately fifty percent or more, is fixed in place, such as to the bottom of the housing 402, so that the reservoir 404 can collapse with minimal folds, wrinkles, or other potential obstacles to deflation and so that the reservoir 404 tends to collapse in a predictable manner and pattern.

In one non-limiting example, the reservoir 404 is defined by first and second oblong panels joined along their edges in a sealed manner to define the shell or outside wall structure of the reservoir 404 with a body portion and a neck. The reservoir 404 in this embodiment is sealed but for an entry and exit orifice in the neck of the reservoir 404, although it would be within the scope of the invention to have separate entry and exit orifices. The shell defining the reservoir 404 is formed from a flexible and substantially gas impermeable material. One of skill in the art may be aware of numerous such materials after reviewing the present disclosure, each within the scope of the invention.

The decoupling reservoir 404 thus comprises a shell of flexible material that acts as an envelope for retaining a volume of oxygen at ambient pressure throughout the entire breathing cycle simply waiting to be naturally inhaled by the patient. Based on its structural properties as taught herein, including one or more of the material, material thickness, and structural configuration of the shell, and the performance deriving therefrom, the shell of the decoupling reservoir 404 and the decoupling reservoir 404 itself provide no or substantially no resistance to inflation when receiving oxygen from a source 406. Moreover, the decoupling reservoir 404 provides no or substantially no resistance to deflation when oxygen is inhaled therefrom by a patient. When maintained at ambient pressure and without pressurization as taught herein, the material of the shell of the decoupling reservoir 404 and the decoupling reservoir 404 itself are incapable of acquiring or exhibiting elastic potential energy. As such, within the bounds of ambient pressure, a decoupling reservoir 404 that is deflated will not tend to resiliently spring and expand toward an expanded configuration, and a decoupling reservoir 404 that is inflated will not tend to resiliently spring and contract toward a retracted or collapsed configuration. As disclosed herein, the entire shell of the decoupling reservoir 404 can be formed of such a material that is incapable of acquiring or exhibiting elastic potential energy, or the decoupling reservoir 404 could have one or more rigid or other shell portions joined with one or more shell portions formed of such a material that is incapable of acquiring or exhibiting elastic potential energy. The reservoir 404 can thus have combinations including one or more flexible walls, rigid walls, compressible walls, collapsible walls, expandable walls, thin walls, or other walls capable of keeping a volume gas inside.

The shell of the reservoir 404 could, for example, be formed from a foil of flexible polymeric material with or without a lining layer. The material defining the reservoir 404 could, for instance, comprise a foil formed by one or more layers of polymeric material with or without a metallic lining, such as a film of aluminum. Thus, one non-limiting material for the shell of the decoupling reservoir 404 that is incapable of acquiring or exhibiting elastic potential energy at ambient pressure is a lightweight, flexible foil. One such flexible foil is founded on a film of polymeric material, such as a film of polyester, even more particularly a film of biaxially oriented polyethylene terephthalate (BOPET). As used herein, the term “film” shall mean an exceedingly thin layer. To achieve the desired properties of being incapable of acquiring or exhibiting elastic potential energy at ambient pressure while demonstrating sufficient gas impermeability, the film of polymeric material comprising polyester and even more particularly biaxially oriented polyethylene terephthalate preferably has a thickness of less than 50 microns (0.002 inches). Even more preferably, such a film of polymeric material will have a thickness of less than 25 microns (0.001 inches). Ideally, the thickness of such a film of polymeric material has a thickness between 2 and 20 microns (between 0.0000787 inches and 0.000787 inches) with 18 microns (0.000709 inches) being employed in present embodiments of the system 400.

While biaxially oriented polyethylene terephthalate polyester film may be preferable in many circumstances for its tensile strength, toughness, and durability, another possible polymeric film operative as the wall material forming the shell of the decoupling reservoir 404 is low density polyethylene. The low density polyethylene film will again be sufficiently thin as to be incapable of acquiring or exhibiting elastic potential energy at ambient pressure while demonstrating sufficient gas impermeability. A low density polyethylene film with a thickness of less than 100 microns (0.004 inches) is preferred. More preferably, such a film of low density polyethylene film material will have a thickness of less than 25 microns (0.001 inches). Ideally, the thickness of such a film of polymeric material has a thickness between 5 and 20 microns (between 0.000197 inches and 0.000787 inches). While low density polyethylene and biaxially oriented polyethylene terephthalate polyester polymeric films are preferred, it will be understood that other polymeric films could be employed in practices of the invention except as may be expressly excluded by the claims.

The film of polymeric material forming the shell of the decoupling reservoir 404 can be metalized, such as with an aluminum film lining, thereby further reducing the permeability of the film. In one non-limiting practice, a film of aluminum or potentially another metal can be evaporated onto the film of polymeric material. For avoidance of doubt, the term “foil” as used herein shall not be interpreted to require metallization except as expressly set forth.

As is enabled by formation of the reservoir 404 of a lightweight, flexible polymeric film or other material incapable of acquiring or exhibiting elastic potential energy, the reservoir 404 once expanded tends even when it is at ambient pressure to substantially maintain an expanded shape and configuration, whether by its own structural integrity or otherwise, even when it is open to external ambient pressure, such as by a fluidic connection to the recipient 426 through ambient pressure tubing 422. As taught herein, when expanded, the reservoir 404 in preferred embodiments does not significantly collapse on its own due to the weight of its walls.

When filled with oxygen, the reservoir 404 thus temporarily stores a compartmented volume of oxygen at ambient pressure waiting to be drawn therefrom by the recipient 426 without spontaneously collapsing and exhausting oxygen therefrom. Concomitantly, again by virtue of being formed from a lightweight, flexible film or other material incapable of acquiring or exhibiting elastic potential energy at ambient pressure, the decoupling reservoir 404 provides substantially no resistance to collapse as the oxygen therein is naturally inhaled by the recipient 426. A patient can thus naturally inhale without opposition from the decoupling reservoir 404 as would exist, for instance, with a reservoir exhibiting elastic potential energy as would be the case with, for example, a resilient rubber bladder exhibiting elastic potential energy.

A fluidic connector 418, which in this example comprises a T-shaped connector, has a first, longitudinal port in fluidic communication with the donor reservoir 404, such as through the aperture in the neck 470 of the reservoir 404. The fluidic connector 418 has a second, longitudinal port in fluidic communication with the ambient pressure tubing 422 and, through that tubing 422, the recipient 426. Finally, the fluidic connector 418 has a third, lateral port between the first and second openings in fluidic communication with the oxygen source 406. The fluidic communication from the source 406 to the connector 418 could, for instance, be through high-pressure tubing 408 acting as a supply conduit from the oxygen source 406 to an oxygen connector 410 fixed to the housing 402 and high-pressure tubing 452 from the oxygen connector 410 to a supply valve 412. At least the first and second ports are in fluidic communication with one another within the fluidic connector 418.

The supply valve 412, which in this example comprises an electromechanical solenoid valve 412, has an open condition and a closed condition. The valve 412 is fluidically interposed between the pressurized oxygen source 406 and the reservoir 404. When the supply valve 412 is in the open condition, oxygen can be passed from the oxygen source 406, through the tubing 408, through the valve 412, through the connector 418, and into the reservoir 404. When the valve 412 is in the closed condition, the passage of oxygen between the oxygen source 406 and the reservoir 404 is prevented.

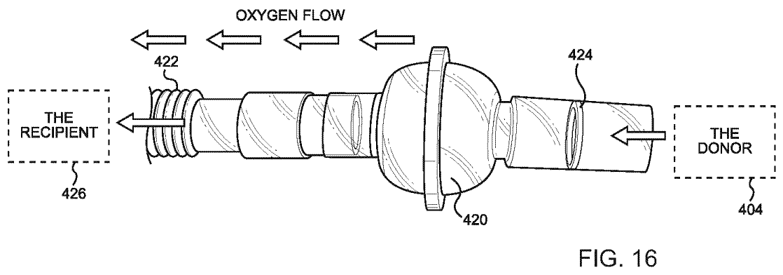

A one-way inspiratory valve 424 is interposed between the reservoir 404 and the recipient 426, such as by being fluidically connected to the second port of the fluidic connector 418 and with the fluidic connector 418 fluidically connected through its first port to the neck 470 of the reservoir 404. The one-way inspiratory valve 424 is operative to enable gas to flow from the donor reservoir 404, through the ambient pressure tubing 422, and to the recipient 426 but to prevent reverse gas flow, such as from the recipient 426 and into the donor reservoir 404. A gas filter 420 is fluidically interposed between the recipient 426 and the one-way inspiratory valve 424 and thus between the recipient 426 and the donor reservoir 404. The filter 420 and the one-way inspiratory valve 424 are shown apart from the remainder of the system 400 in FIG. 16.

As disclosed herein, the volume of oxygen in the decoupling reservoir 404 is retained substantially at ambient pressure. Ambient pressure can be defined as the pressure of the air surrounding the decoupling reservoir 404. By operation of the present system 400, the decoupling reservoir 404 is not maintained in, indeed not inflated to, a state of pressurization. As a recipient 426 performs the inspiratory phase of breathing, oxygen will be drawn from the decoupling reservoir 404 through the ambient pressure tubing 422 thereby drawing from and tending to reduce the volume of oxygen in the decoupling reservoir 404. Due to the compressible nature of the decoupling reservoir 404 without elastic potential energy in the walls forming the shell thereof, the reservoir 404 will tend to contract without imparting resistance to the natural inhalation of the patient. When the reservoir 404 contracts by a sufficient amount, the reservoir 404 is automatically replenished with oxygen by operation of an inflation detection system without pressurization of the reservoir 404 so that the oxygen within the reservoir 404 remains substantially at ambient pressure.

The inflation detection system has a first condition wherein replenishing oxygen is not supplied to the decoupling reservoir 404 and a second condition wherein replenishing oxygen is supplied to the decoupling reservoir 404. The first condition can be a condition wherein the donor reservoir 404 is inflated with oxygen to a certain, predetermined maximum state of inflation, and the second condition can be a condition wherein the donor reservoir 404 is inflated with oxygen below a predetermined minimum state of inflation. With the decoupling reservoir having a fully inflated condition, the predetermined maximum state of inflation is less than that fully inflated condition. The decoupling reservoir can be considered to have a fully deflated condition with the predetermined minimum state of inflation being less than the predetermined maximum state of inflation but greater than the fully deflated condition.

The inflation detection system is operative to detect when the donor reservoir 404 has reached the predetermined maximum and minimum states of inflation. The predetermined maximum state of inflation can be detected when the decoupling reservoir 404 reaches a predetermined size or other inflation condition in any dimension or combination of dimensions. In embodiments of the invention, the decoupling reservoir 404 can be considered to have a fully inflated condition, and the inflation detection system detects when the decoupling reservoir 404 is inflated to the fully inflated condition or to within a predetermined range of the fully inflated condition. By way of example and not limitation, the inflation detection system can detect when the decoupling reservoir 404 is inflated with oxygen at or above a maximum threshold inflation level, which may be equal to or less than the fully inflated condition.

Made aware of the present invention, one skilled in the art may appreciate plural mechanisms that would operate as inflation detection systems to detect when the decoupling reservoir 404 is inflated to the predetermined state of inflation. Each such mechanism is within the scope of the invention except as it may be expressly limited by the claims. Inflation detection mechanisms could comprise mechanical systems, electrical systems, electromagnetic systems, optical systems, electro-mechanical systems, sound-activated systems, movement sensors, light sensors, and any other type of system effective to detect when the reservoir 404 is inflated to a predetermined state of inflation with it again being noted that the predetermined state of inflation is reached while the oxygen within the decoupling reservoir 404 is substantially at ambient pressure.

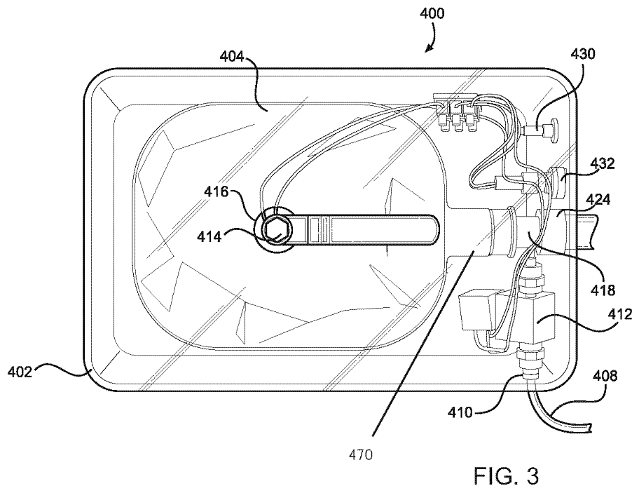

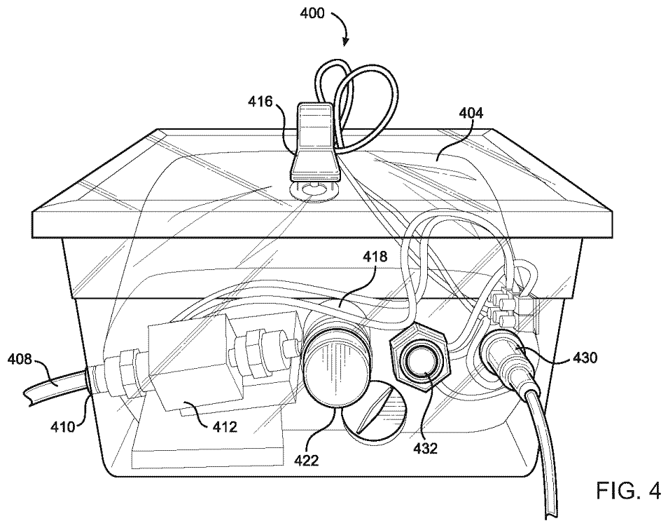

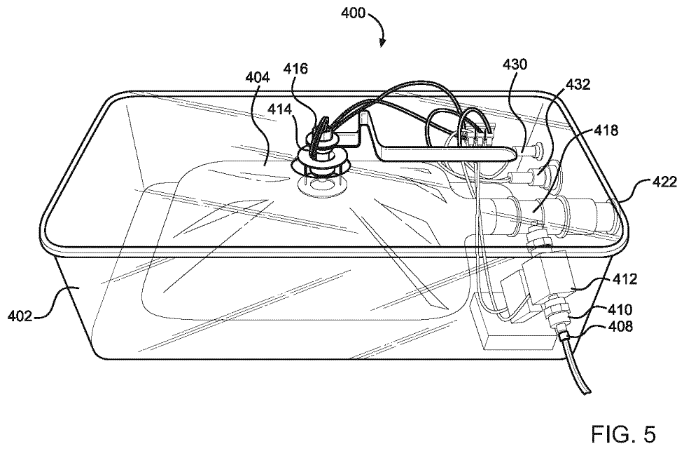

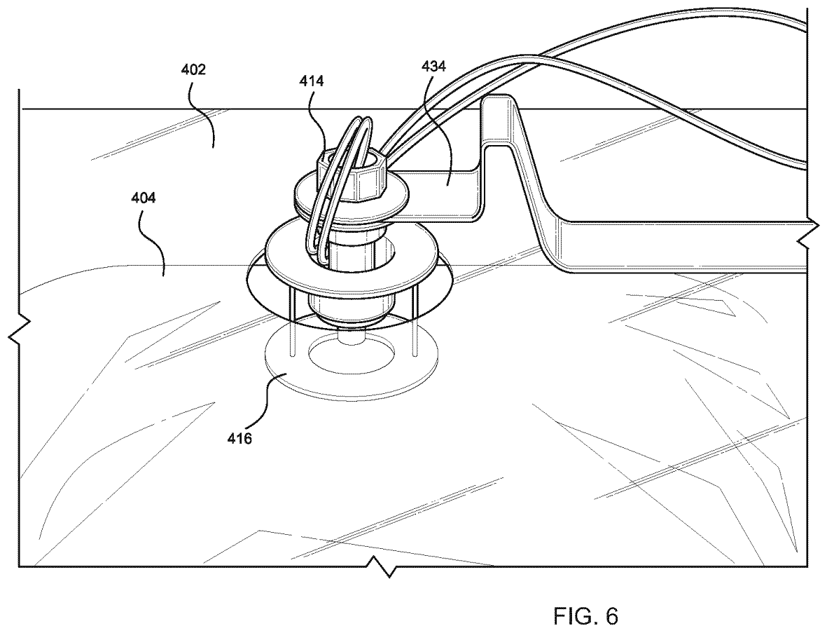

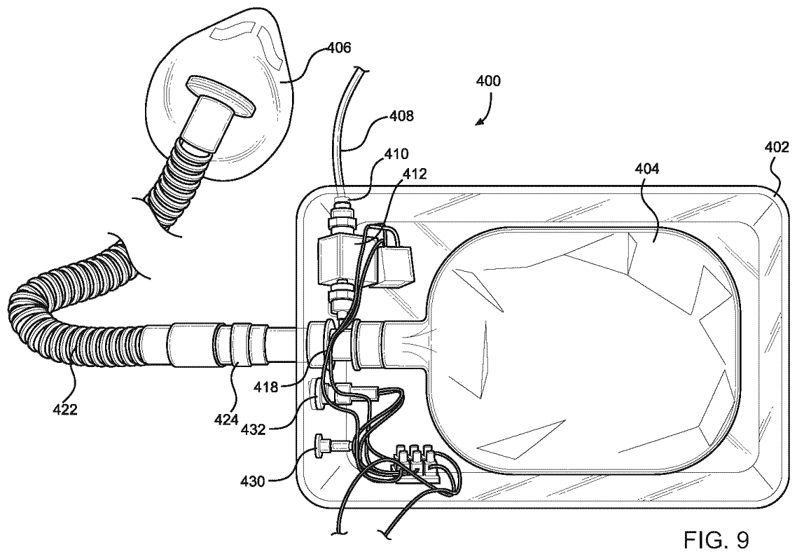

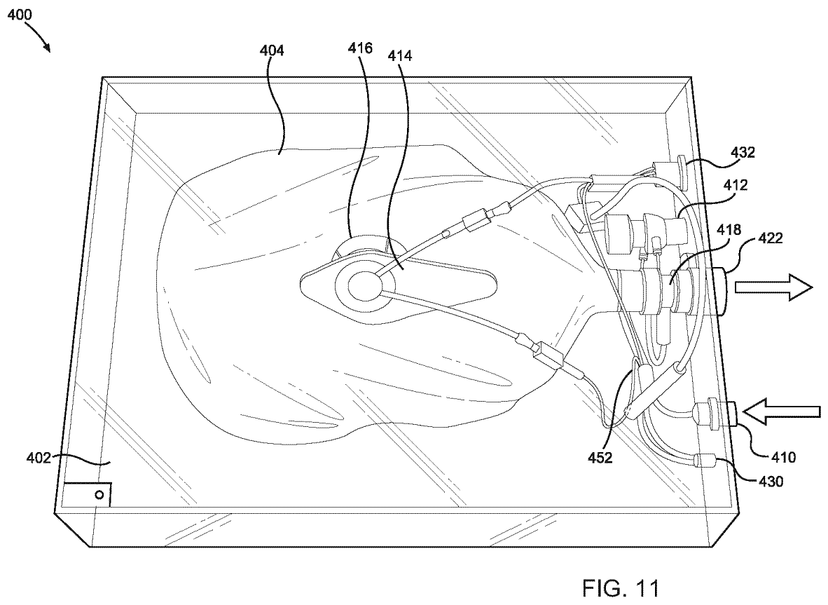

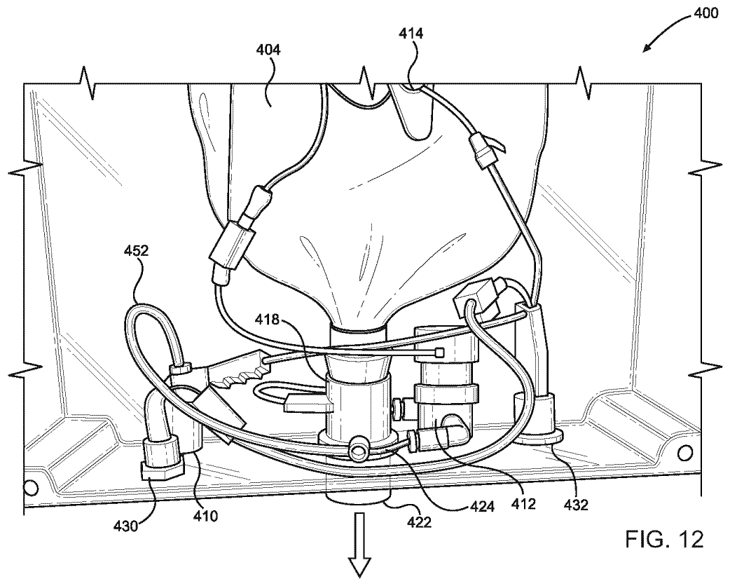

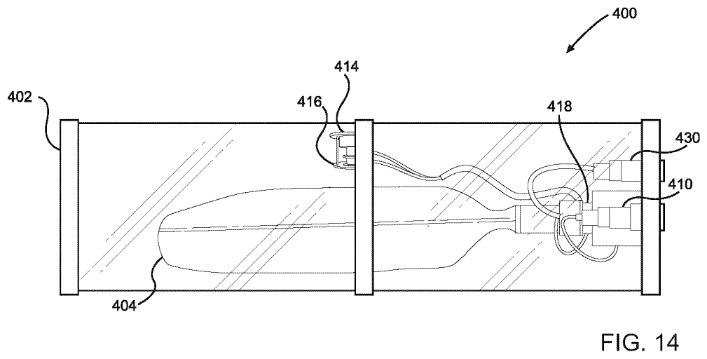

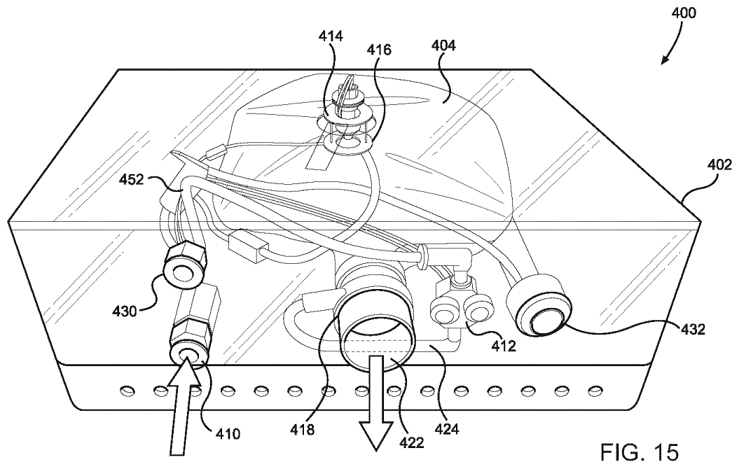

In the non-limiting embodiment of FIG. 1, the inflation detection system comprises an electro-mechanical system for detecting when the decoupling reservoir 404 is filled to the predetermined state of inflation. The inflation detection system has a contact structure 416 disposed to contact, to be contacted by, to be moved by, or otherwise to be actuated by the decoupling reservoir 404 when the reservoir 404 reaches a stage of inflation. Within the scope of the invention, the location and construction of the contact structure 416 could vary. In the embodiment of FIG. 1, for instance, the contact structure 416 is disposed to project from or through the distal end wall of the housing 402 and into the inner volume of the housing 402 so that it projects toward and can engage the distal end of the reservoir 404. In the embodiments of FIGS. 3 through 15, however, the contact structure 416 is disposed to project from or through the upper wall of the housing 402 and into the inner volume of the housing 402 to engage a mid-portion of the reservoir 404. There, the contact structure 416 is retained by a support structure 434 that is fixed to the upper wall of the housing 402. According to the invention, the contact structure 416 could be otherwise retained.

The contact structure 416 is positioned to be moved by the reservoir 404 as the reservoir 404 expands toward an inflated condition. The contact structure 416 can, for instance, be depressed, pivoted, rotated, or otherwise actuated by the reservoir 404 and more particularly by an expansion of the reservoir 404. The contact structure 416 operates as or as a component of or to actuate a flow switch 414. When the contact structure 416 is actuated by the expansion of the reservoir 404, the flow switch 414 is caused to actuate the valve 412 from the ON condition where oxygen is permitted to flow from the oxygen source 406 to the reservoir 404 to replenish and fill the reservoir 404 to the OFF condition where oxygen is prevented from flowing from the oxygen source 406 to the reservoir 404. The contact structure 416 is biased, such as by spring force, under the force of gravity, by resiliency, or any other biasing method or combination thereof toward the donor reservoir 404.

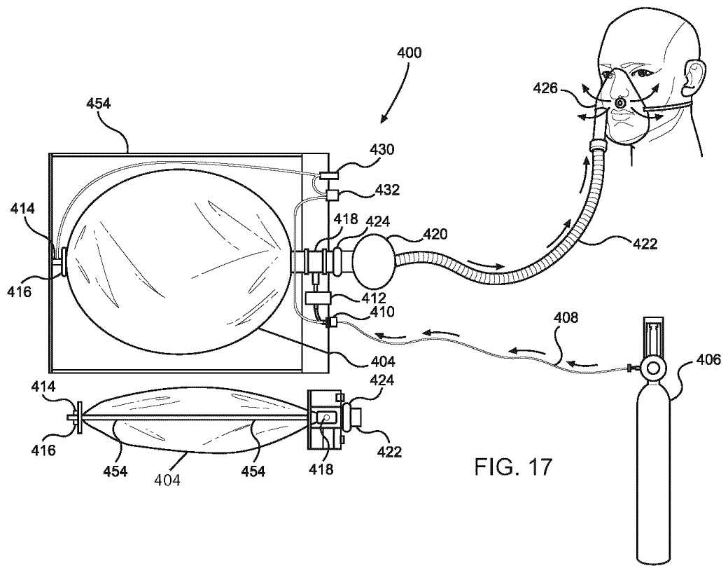

In the embodiment of FIG. 1, the decoupling donor reservoir 404 is disposed within a housing 402. Additionally or alternatively, the reservoir 404 could be disposed within a sub-housing that, in turn, could be disposed in the housing 402 or that could stand independently. Still further, as is shown in FIG. 17, for example, the decoupling reservoir 404 could be disposed without a housing or enclosure, in which case the contact structure 416 and potentially the flow switch 414 described further hereinbelow could be otherwise retained, such as by a surrounding band, a rigid arm, or another retaining structure 454, for contact or other sensing or engagement relative to the donor reservoir 404. The contact structure 416 and the flow switch 414 could be retained together, potentially as a unit, or in separate dispositions. In FIG. 17, the contact structure 416 is retained by the retaining structure 454, which could be a rigid support arm or any other retaining structure, to engage the decoupling donor reservoir 404, and the flow switch 414 is integrated with the contact structure 416.

The contact structure 416 is thus retained by the housing 402, by the retaining structure 454, or otherwise to contact the reservoir 404. Without limiting the invention, the contact structure 416 could be retained to contact the reservoir 404 by being secured partially or entirely within the housing 402 or through an aperture in the housing 402 or through an aperture in a sub-housing that retains the reservoir 404, or the contact structure 416 could be retained to contact a decoupling donor reservoir 404 that is not in a housing at all.

The flow switch 414 has an activated state, which may be considered to be the ON condition, when the contact structure 416 is sufficiently moved, such as by extension, pivoting, or other movement, in an inward direction toward the inner volume of the decoupling donor reservoir 404. The contact structure 416 is permitted to move inwardly in the direction toward the donor reservoir 404 to the activated state when the volume of oxygen in the donor reservoir 404 falls below the predetermined state of inflation such that the outside wall is or can be deflected or moved inwardly. The flow switch 414 has a deactivated state, which may be considered to be the OFF condition, when the contact structure 416 is moved, such as by retraction, pivoting, or other movement in an outward direction away from the donor reservoir 404. The contact structure 416 is moved outwardly to adjust the flow switch 414 to the deactivated state, which is the OFF condition, when the volume of oxygen in the decoupling donor reservoir 404 reaches the predetermined state of inflation to cause the outside wall of the decoupling donor reservoir 404 to be advanced outwardly by the expansion of the decoupling donor reservoir 404. For instance, where the contact structure 416 is a depression switch, expansion of the reservoir 404 will press the outer wall or shell of the reservoir 404 outwardly to press the contact structure 416 and the flow switch 414 to the deactivated state.

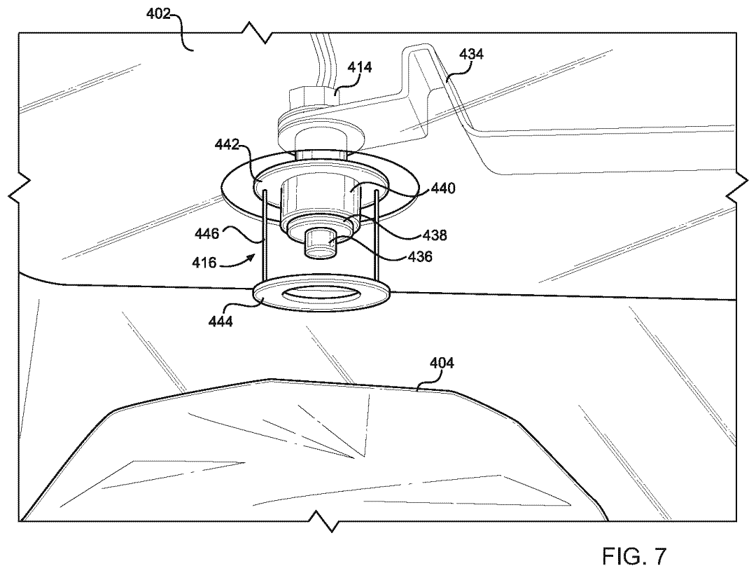

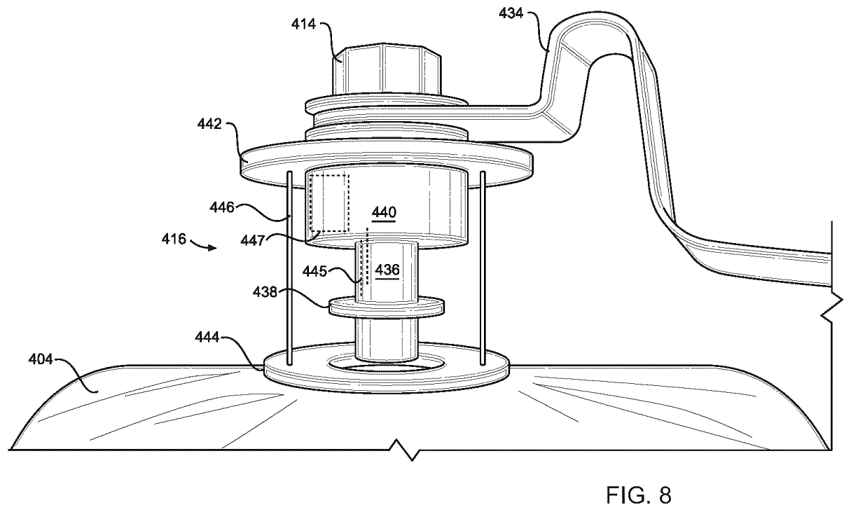

In the embodiment of FIGS. 3 through 10, the contact structure 416 and the switch 414 are embodied as a float switch with an actuation framework. With particular reference to FIGS. 7 and 8, the actuation framework of the contact structure 416 can be seen to be retained to be movable along a vertical axis generally perpendicular to a longitudinal of, and generally the surface of, the decoupling donor reservoir 404. The actuation framework of the contact structure 416 has a distal, flat toroidal ring 444 disposed to engage and be engaged by the wall of the reservoir 404. A proximal toroidal ring 442 is maintained in parallel spaced relation to the distal toroidal ring 444 by a plurality of rod members 446, and a collar 440 is fixed to move with the proximal toroidal ring 442.

The actuation framework so formed by the toroidal rings 442 and 44, the rod members 446, and the collar 440 is extendable and retractable relative to a central column 436. An annular plate 438 is fixed along the length of the central column 436 distal to the collar 440 of the actuation framework so that the annular plate 438 retains the contact structure 416 in a floating manner. As FIG. 7 shows, the collar 440 tends to drop into contact with the annular plate 438 under the natural force of gravity when the reservoir 404 is filled below a given level.

The central column 436 houses a magnetic switch 414, such as a reed switch 414, and the floating actuation framework of the contact structure 416 retains a magnet 447 within the collar 440. When the actuation framework is extended as in FIG. 7 where the decoupling donor reservoir is below the state of inflation, the contacts 445 of the reed switch 414 (shown in FIG. 8) are attracted into contact with one another to complete the electrical circuit and trigger the switch 414 to an activated condition, which actuates the valve 412 to the ON condition where oxygen is permitted to flow from the oxygen source 406 to the reservoir 404. When the reservoir 404 is filled to the predetermined state of inflation, the magnet 447 within the collar 440 is moved away from the contacts 445 of the reed switch 414 to break the circuit and trigger the switch 414 to a deactivated condition, which actuates the valve 412 to the OFF condition wherein oxygen is prevented from flowing from the oxygen source 406 to the reservoir 404.

By virtue of the biasing of the contact structure 416, which can be by any mechanism including gravity, a resiliently compressible or extendible member, or any combination of mechanisms, the contact structure 416 automatically moves to the activated state to actuate the flow switch 414 and the valve 412 to the ON condition to permit oxygen to flow from the oxygen source 406 to the reservoir 404 when the volume of oxygen in the donor reservoir 404 falls below the predetermined threshold value, such as below the predetermined minimum state of inflation. When the volume of oxygen in the donor reservoir 404 reaches the predetermined threshold value, such as at or above the predetermined maximum state of inflation, the contact structure 416 is moved by the wall of the decoupling donor reservoir 404 to the deactivated state, and the switch is disposed in the OFF condition. In the deactivated state, the valve 412 is closed to prevent the flow of preservative gas from the source 406 to the donor reservoir 404.

The decoupling reservoir 404 can thus be inflated, such as to or within a given range of the maximum volume of the reservoir 404 without over-inflation or pressurization of the reservoir 404. Oxygen within the reservoir 404 is thus prevented from exceeding approximately ambient pressure. Except as might otherwise be required by the claims, however, embodiments of the invention could calibrate the contact structure 416 or the flow switch 414 or both to be induced to the deactivated state at some other predetermined inflation condition or pressure, including potentially a pressure or inflation condition in excess of ambient pressure or to some inflation condition well below the maximum volume of the decoupling donor reservoir 404. The flow switch 414 and the valve 412 can be electrical, mechanical, electro-mechanical, or otherwise configured and constructed.

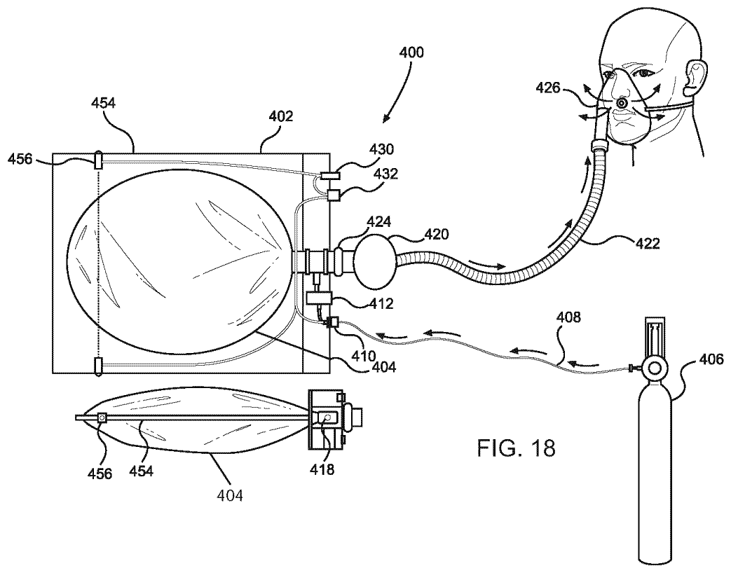

It will again be observed that one skilled in the art would appreciate other mechanisms that would operate as inflation detection systems to detect when the decoupling reservoir 404 is inflated to the predetermined state of inflation with each such mechanism being within the scope of the invention except as it may be expressly limited by the claims. For instance, as in the embodiment of FIG. 18, for instance, the inflation detection system could alternatively take the form of a contactless detection system 456, such as an optical detection system that could be carried forth by, for instance, a laser detection system, a camera system, an infrared inflation detection system, or any other effective optical or contactless detection system. In the embodiment of FIG. 18, for example, a contactless detection system 456 is formed with a light emitter, such as a laser or other light emitter, retained to one side of the reservoir 404 and a light receptor disposed to the opposite side of the reservoir 404. Under such constructions, the inflation condition of the reservoir 404 can be sensed in a contactless manner, such as based on a distance of a wall surface of the reservoir from a sensor of the contactless detection system 456 or where the reservoir 404 is inflated to a condition where the reservoir 404 prevents the communication of light from the light emitter to the light receptor, where the reservoir 404 demonstrates a predetermined reflectance value, or in some other contactless manner.

In the embodiments of the automatic gas conservation system 400 of FIGS. 1 through 16, the supply valve 412 comprises a solenoid valve that is in electrical communication, such as through electrical wiring in an electrical circuit, with the flow switch 414. As illustrated, an electrical control system, which can include electrical circuitry, electronic memory, wiring, and other electrical control and connection components, cooperates with the inflation detection system to induce the solenoid supply valve 412 to an open condition to permit the flow of oxygen from the source 406 when the flow switch 414 is in the activated state. The electrical control system can receive power from a power source, which could be a source of alternating current through a power supply connection 430, a source of direct current such as a battery power source, or some other source of electric power. The flow of electrical power from the power source can be controlled by a power switch 432. The solenoid valve 412 is induced by the inflation detection system and the electrical control system to a closed condition to prevent the flow of oxygen from the source 406 to the reservoir 404 when the flow switch 414 is in the deactivated state. Each of the components referenced herein can be further combined or separated within the scope of the invention.

The solenoid valve 412 can be electrically opened when the electrical circuit is closed by the movement or other actuation of the flow switch 414 of the inflation detection system to the activated condition. The solenoid valve 412 is automatically closed to prevent further filling of the donor reservoir 404 when the electrical circuit is opened by the contact structure 416 and the flow switch 414 is moved to the deactivated condition, which can be indicative that the donor reservoir 404 is filled to the predetermined state of inflation. In the example of the invention where the contact structure 416 and the flow switch 414 are actuated by a pressing or pushing of the contact structure 416 outwardly by the reservoir 404, an open electrical circuit is established where no electricity flows when the contact structure 416 is sufficiently pressed outwardly by the reservoir 404 and the solenoid valve 412 is in a closed position. When the contact structure 416 is sufficiently advanced, such as by extension inwardly toward the reservoir 404, indicating that the reservoir 404 has fallen below the predetermined state of inflation, the electrical circuit is closed to permit the flow of electricity to actuate the solenoid valve 412 to an open condition so that oxygen can flow to fill the donor reservoir 404.

Even when the valve 412 is in an open condition, the rate of flow, the pressure of flow, or both the pressure and rate of flow of oxygen from the source 406 to the donor reservoir 404 can be limited, such as by a flow-limiting connector 415 as shown, for instance, in FIG. 1. The flow-limiting connector 415 could limit the flow rate of oxygen from the source 406 to the donor reservoir 404 to a predetermined flow rate, such as less than 1 liter per minute or any other flow rate. The flow-limiting connector 415 could, for example, comprise a narrow-diameter tube connector, such as a connector having an inner diameter of 0.02 mm or some other dimension reduced as compared to other conduit connections within the fluidic system. Rapid changes in pressure within the donor reservoir 404 can thus be prevented on opening of the valve 412.

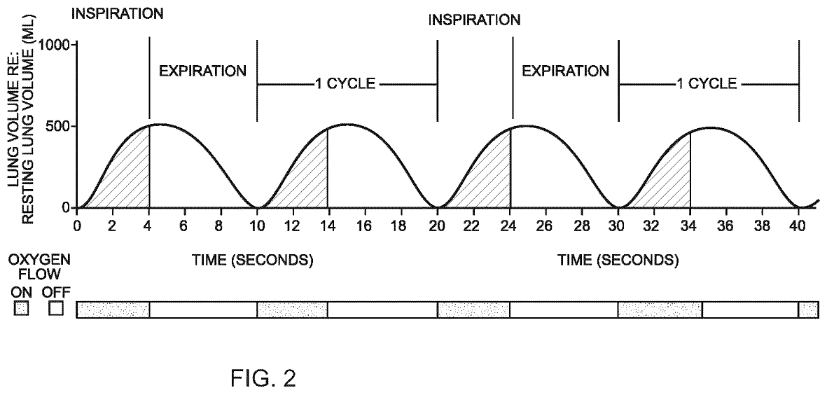

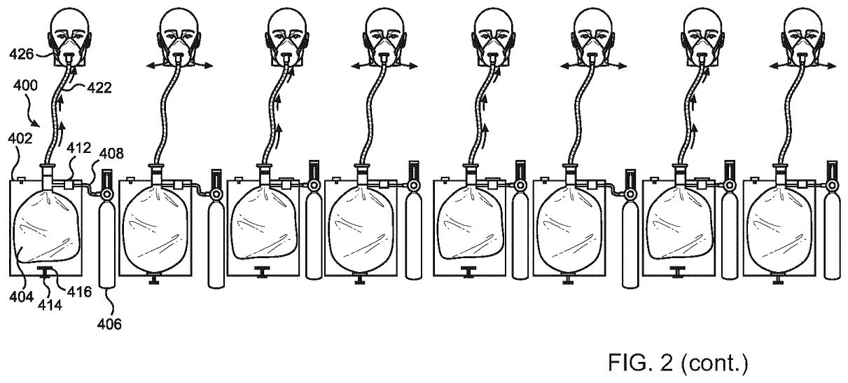

Referring to FIG. 2, the system 400 for the decoupled supply and conservation of oxygen and other substances, which may alternatively be referred to as the automatic gas conservation system 400, is depicted in operation during a series of respiratory cycles to provide an on-demand supply to a recipient 426, such as a mask worn by a human or other living patient in need. In operation of the automatic gas conservation system 400, natural inspiration by the patient will operate to draw oxygen at ambient pressure from the decoupling donor reservoir 404 thereby tending to contract the reservoir 404. When the reservoir 404 falls between the predetermined minimum state of inflation, the reservoir 404 is automatically filled to the predetermined maximum state of inflation by a supply of oxygen from the source 406. A volume of continually-replenished oxygen at ambient pressure is thus available within the reservoir 404 to be drawn through the one-way inspiratory valve 424 and the ambient pressure tubing 422 during a natural inspiration phase of a breathing cycle. When the recipient 426 is not engaged in inspiration, no oxygen is drawn from the reservoir 404. When the volume of oxygen within the reservoir 404 falls below the predetermined minimum state of inflation, the inflation detection system formed by the contact structure 416 and the flow switch 414 or other inflation detection system will detect the same and trigger the valve 412 to an open condition. Flow of oxygen is then permitted from the oxygen source 406 so that the donor reservoir 404 will be filled with oxygen until the predetermined maximum state of inflation is reached. When the predetermined maximum state of inflation is reached, the inflation detection system will detect the same and trigger the valve 412 to a closed condition to prevent the further supply of oxygen to the donor reservoir 404 from the source 406 until a further inspiration phase of a breathing cycle draws a volume of oxygen from the reservoir 404.

The donor reservoir 404 is thus automatically supplied with oxygen while pressurization of the oxygen in the reservoir 404 is automatically prevented. Supplemental oxygen is safely and effectively supplied to the patient by being made available in the envelope defined by the reservoir 404 at ambient pressure in an on-demand volumetric displacement system enabling the transfer of oxygen during the entire inspiratory phase of the breathing cycle while the wasteful release of oxygen during the expiratory phase of breathing, indeed at any phase other than the inspiratory phase, is prevented.

The donor reservoir 404 can be caused to receive replenishing oxygen from the pressurized source 406 through the high-pressure tubing 408 and through the supply valve 412 automatically as soon as the reservoir 404 begins to collapse or as soon as the reservoir 404 deflates to the predetermined minimum state of inflation. The automatic refilling of the reservoir 404 ensures that the donor reservoir 404 always retains a supply of oxygen available for the next inspiratory phase of the breathing cycle while the oxygen in the reservoir 404 never exceeds ambient pressure. Where the donor reservoir 404 is visually exposed, such as through a partially or completely transparent housing 402 or an observation aperture in the housing 402, an observer is provided with visual confirmation of the state of inflation of the donor reservoir 404. The automatic gas conservation system 400 can thus provide a synchronized delivery of supplemental oxygen to a recipient 426 as the donor reservoir 404 and the system 400 in general synchronize with the physiological ventilations of a patient based on the storage and replenishment of oxygen in the donor reservoir 404 at ambient pressure and the termination of the supply of oxygen automatically on the donor reservoir 404 reaching the predetermined state of inflation. Advantageously, the process of inflating the reservoir 404 is decoupled from the process of inhalation by the patient such that oxygen at ambient pressure in the decoupling reservoir 404 is rendered available for natural inhalation while the reservoir 404 is automatically reinflated when a minimum state of inflation is reached.

Within the scope of the invention, the system 400 can measure, record, and analyze the flow of oxygen and the breathing characteristics of a patient. By way of non-limiting example, a volumetric measuring flow meter could be connected to the source 406 of oxygen. Additionally or alternatively, one or more flow meters could be retained within the housing 402 along the path of gaseous flow through the system 400. For instance, a flow meter could be disposed to measure oxygen passing through the valve 412. In the depicted embodiments, the valve 412 can incorporate a flow meter such that the same should be considered to be illustrated therewithin, or a flow meter could be otherwise disposed. For instance, a flow meter could further or alternatively be disposed between the reservoir 404 and the ambient pressure tubing 422. By measuring the volume of oxygen supplied to a recipient 426 by the system 400, such as over a given time period, per cycle of inspiration and expiration, or otherwise, plural determinations, measurements, and analyses can be made. For instance, one can determine the volume of oxygen inspired by the patient and, additionally or alternatively, the volume of oxygen remaining in the oxygen source 406. Through electronic memory and software operating on the electrical system or in communication therewith, the system 400 can harvest, process, and analyze data based on usage of the system 400.

As often shown and described herein, the recipient 426 can be the breathing mask of a living patient receiving supplemental oxygen, but other recipients and delivery equipment are possible and within the scope of the invention. When worn by a patient, the patient and the breathing mask or other oxygen delivery equipment may collectively be referred to as the recipient 426. Other recipient delivery equipment could, for example, comprise other respiratory accessories, such as but not limited to nasal cannulas, laryngeal mask airways (LMA), endotracheal tubes, tracheotomies, ventilator attachments, CPAP machine connectors, Ambu bags, or even delivery devices for recreational oxygen. The automatic gas conservation system 400 is not limited with respect to the recipient 426 unless the claims expressly so require.

As shown in FIG. 1, a recipient mask 426 can have one or more one-way expiratory valves 428 and can include adjustment mechanisms as is known in the art for adjusting oxygen supply to the patient. As necessary, the concentration of oxygen that the patient needs as determined by the physician can be reliably and predictably diluted and controlled with devices currently in use and that are within the scope of the system 400. By way of example and not limitation, the number, diameter, or other characteristic of orifices in the inspiration tube 422 or the recipient mask 426 can be adjusted to allow more or less oxygen to achieve the desired concentration to the recipient mask 426 for the patient as clinically needed.

With further reference to FIG. 2, the method for the necessary supply of oxygen to a patient recipient 426 and the synchronized operation of the automatic gas conservation system 400 in relation thereto can be further understood. There, the dynamics of the breathing cycle are depicted in parallel with the filling and refilling operations of the donor reservoir 404 of the automatic gas conservation system 400. To expand the lungs, the inspiratory muscles overcome two key factors, namely, compliance of the lungs and airway resistance mainly in the form of frictional resistance to the flow of air through the airways. At the start of inspiration, the diaphragm contracts and descends, expanding the thoracic volume. The descent of the diaphragm compresses the abdominal contents and decompresses the contents of the thoracic cavity. With expansion of the thoracic cavity and its decompression, both intrapleural pressure and alveolar pressure decrease. Alveolar pressure decreases to a sub-atmospheric level, and the pressure gradient for the flow of air into the lungs is established. Air flows into the lungs and lung volume increases until the alveolar pressure rises to the atmospheric level (0 cm H₂O) when the pressure gradient for flow of air into the lungs ceases to exist. At the end of quiet inspiration, intrapleural pressure reaches about −8 cm H₂O, and the transpulmonary pressure distending the lungs increases to 8 cm H₂O (Pl=Pa−Ppl=0−(−8)=8 cm H₂O).

During quiet expiration, the cycle is reversed. The inspiratory muscles relax, and the inward elastic recoil of the lungs results in deflation of the lungs. During deflation, the lungs and chest wall move as one unit. Airflow out of the lungs ceases when alveolar pressure equals atmospheric or ambient pressure (0 cm H₂O).

Based on Boyle's law, in a closed system where the number of gas molecules is constant, at any constant temperature, the pressure exerted by a gas varies inversely with the volume of the gas. Therefore, as the volume of a gas increases, the pressure exerted by the gas decreases. Conversely, the pressure increases as the volume decreases.

Accordingly, in operation of the present system 400 and method, when a patient takes a breath during the inspiratory phase of the breathing cycle, a continuous flow of supplemental oxygen enters the patient's lungs from the system 400 throughout the entire inspiratory phase of the breathing cycle. The flow rates, pressures, and volumes are different at different points of the inspiratory phase. The flow starts by a drop in alveolar pressure below ambient pressure inside the donor reservoir 404 with it being again recognized that the system 400 could work with higher and lower pressures than ambient unless the claims require otherwise. Then, the donor reservoir 404 supplies non-pressurized oxygen at ambient pressure directly to the patient through the recipient 426 as a continuous flow but at different speeds during the inspiratory cycle. While the process of inflation and inhalation are decoupled, the flow rates, pressures, volumes, and respiratory rate are effectively synchronized to those of the patient due to the donor reservoir 404 being maintained at ambient pressure. In other words, the processes of inhalation and inflation are decoupled while the supply of oxygen is automatically matched or synchronized to the patient's particular needs. The speed, pressure, time, and volume of patient inspiration and gaseous transfer from the donor reservoir 404 are equivalent and will likely be substantial mirror images. Having a system 400 that matches the supplement of oxygen to a patient's physiological ventilation values at each point in time throughout the inspiratory phase of the breathing cycle ensures reliable delivery of the prescribed oxygen concentration through a recipient facemask 426 or any other oxygen delivery equipment available without supplementing less or more oxygen flow than planned. Flow rate, alveolar pressure, and tidal volume can be synchronized at each point throughout the inspiratory phase of the breathing cycle with it being recognized that the physiological ventilation values of patients are different at different points in the inspiratory phase.

During inhalation, a continuous flow of oxygen towards the patient's lungs is made available until the patient's intrathoracic pressure is at equilibrium with the ambient pressure of the donor reservoir 404 at the end of the inspiratory phase of the breathing cycle. At that time, as in natural breathing, the flow of oxygen to the patient stops until the beginning of the next inspiratory phase. Since there is no demand by the patient, no oxygen flows from the system 400 to the patient during the expiratory phase of the breathing cycle, but a flow of oxygen from the source 406 of compressed, high-pressure oxygen is supplied in a decoupled process to the cause the donor reservoir 404 to expand until the predetermined maximum state of inflation is reached. Once the reservoir 404 is refilled to the predetermined maximum state of inflation and at ambient pressure, the donor oxygen reservoir 404 is ready to supply supplemental oxygen when the patient's next inspiratory phase begins. The inflation detection system automatically shuts off the supply valve 412 to prevent further oxygen flow once the reservoir 404 reaches the predetermined maximum state of inflation while remaining at ambient pressure. The passive and sustained supply of a reliable volume and concentration of supplemental oxygen from the donor reservoir 404 to the patient's lungs throughout the entire inspiratory cycle is thus possible with the donor reservoir 404 placed between a compressed oxygen source 406 and the patient's oxygen delivery equipment, such as a recipient mask 426.

The automatic gas conservation system 400 can thus be employed to provide supplemental oxygen to patients in a wide variety of circumstances. Furthermore, except as the claims may be expressly limited, the automatic gas conservation system 400 is not limited to handling oxygen, and it is not necessarily limited to providing gas to patients at all. Other applications where the dispensing of gas or other substances with automatic replenishment of a reservoir 404 are possible.

It will be understood that many conditions may require supplemental oxygen. For instance, during the pandemic deriving from the COVID-19 coronavirus disease, many thousands of patients required supplemental oxygen due to acute hypoxemic respiratory failure. Other illnesses requiring supplemental oxygen include acute exacerbations of chronic obstructive pulmonary disease (COPD) and acute severe bronchial asthma. Patients with chronic obstructive pulmonary disease often have chronic hypoxemia with or without CO₂ retention. Oxygen in this situation is required until the exacerbation is settled. While a high FiO₂ of up to 100% can be initially administered in case hypoxemia is severe, it is soon tapered to around 50-60% FiO₂. The goal of supplemental oxygen is to maintain a PaO₂ (Partial Pressure of arterial Oxygen) of 55-60 mm Hg, which corresponds to SpO₂ of about 90%. Higher concentrations of oxygen blunt the hypoxic ventilatory drive, which may precipitate hypoventilation and CO₂ retention. It is considered preferable to use a regulated flow device such as a venti mask, which guarantees oxygen delivery to a reasonable extent. Once the patient is stabilized, one can shift to nasal prongs, which are more comfortable and acceptable to most patients. Patients with acute severe asthma or status asthmaticus have severe airway obstruction and inflammation. They are generally hypoxemic. With such conditions, an arterial blood sample is immediately obtained, and oxygen is started via nasal cannula or preferably via a facemask at a flow rate of 4-6 L/min to achieve FiO₂ of 35 to 40%. Higher flow is unlikely to improve oxygenation. The flow rate is adjusted to maintain a PaO₂ of about 80 mm Hg or near normal value. Assisted ventilation is required in case there is persistence of hypoxemia and/or precipitation of hypercapnia.

These clinical samples show the importance of supplying reliable FiO₂ (fraction of Inspired Oxygen) to a patient. However, with conventional systems, they also require continuous flow at high flow rates to overcome air entrapment, making these systems wasteful when supplementing directly compressed oxygen from a cylinder to patients. Also, even if the systems intermittently deliver compressed oxygen only during the inspiratory phase of the breathing cycle, such as with pulse flow (PF), to avoid the continuous delivery of oxygen, these systems must provide pulses of compressed oxygen to the patient containing significant more oxygen than the patient requires to overcome air entrapment.

By providing oxygen only on demand during the inspiratory phase of the breathing cycle and decoupling the processes of inflation and inhalation, the present system 400 and method are elegant and efficient in conserving oxygen and lowering oxygen costs without compromising necessary supply. Since there is no gas delivery to the patient during the expiratory phase of the breathing cycle, the flow of oxygen from the source 406 of compressed oxygen is intermittent during inspiration only and not a continuous flow as demanded by, for instance, high-concentration oxygen masks of the prior art to keep a reliable concentration of oxygen and to overcome air entrapment that otherwise dilutes oxygen concentration and delivers an unreliable concentration to the patient. Oxygen delivery systems using compressed oxygen at a constant flow, particularly at high flow rates, are wasteful and costly. Moreover, the delivery of pressurized oxygen can be complex and difficult, often requiring complicated software, detailed algorithms, and multiple components susceptible to malfunction and breakage thereby requiring repairs and demanding safety mechanisms that further contribute to the cost and complexity of such systems.

Under typical systems of the prior art, a relatively inexpensive oxygen delivery system can be provided, but it demands the constant flow of pressurized oxygen with half or more of the precious gas being simply exhausted to the environment. Systems with oxygen delivered with pulse flow (PF) through a facemask or another oxygen delivery device do seek to supply oxygen only during the inspiratory phase and not during exhalation seeking to reduce total oxygen needs. However, that delivery demands expensive equipment and is not imparted at ambient pressure. Furthermore, providing a pulse of supplemental oxygen properly-timed to synchronize perfectly with the breathing of a patient can be difficult or impossible, particularly where patient oxygen requirements change over time.

The on-demand supply of oxygen to be naturally inspired that is provided by the donor reservoir 404 with the present automatic gas conservation system 400 overcomes numerous deficiencies and limitations exhibited by systems of the prior art. For instance, to achieve the prescribed inspired oxygen concentration, many prior art systems are dependent on the patient's peak inspiratory flow rate (PIFR). For example, when a patient requires low-inspired oxygen concentration, using a nasal cannula at a low flow rate will help, but this practice limits the patient's oxygen only to a low inspired oxygen concentration. Should the patient increase his or her oxygen requirements significantly, the inspiratory effort to drive more air into the lungs, which is dependent on tidal volume, ‘speed’ of inspiration, and respiratory rate, will make the PIFR exceed the flow rate at which oxygen or an oxygen/air mixture is supplied by the nasal cannula or other delivery device. This will mean that at the time of PIFR more or less entrainment of room air occurs, altering the resulting FiO2 in an unpredictable fashion. On the other hand, if high concentrations of oxygen are needed by a patient, using a non-rebreathing face mask at very high flows of oxygen (10-15 L/Min) reassures a reliable delivery of oxygen volume at the prescribe concentration and is less dependent on PIFR. However, half or more of the oxygen is wasted to the environment with supply costs being commensurately increased.

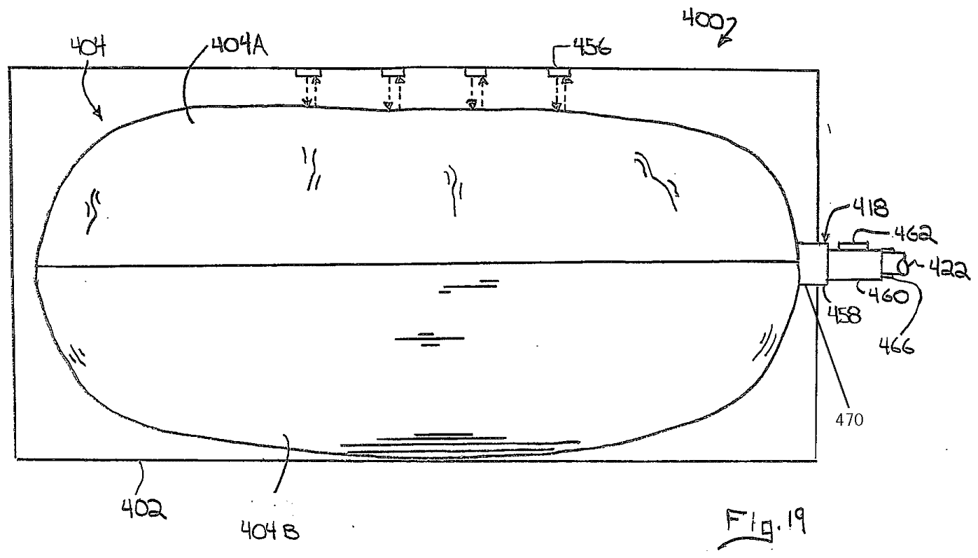

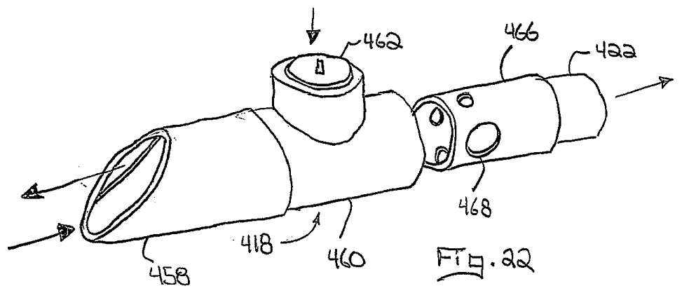

An alternative embodiment of the system for the decoupled supply and conservation of oxygen and other substances 400 is depicted in FIGS. 19 through 21. Components of the system 400 not particularly illustrated in FIGS. 19 through 21 can be as previously shown and described in relation to FIGS. 1 through 18 except as otherwise set forth herein. The system 400 again provides an on-demand supply of oxygen at ambient pressure to a recipient, such as to a patient through a breathing mask 426, from a decoupling donor reservoir 404. The decoupling reservoir 404 retains oxygen at ambient pressure and is continually supplied with oxygen from an oxygen source as previously shown and described. The decoupling reservoir 404 is again disposed within a housing 402, and a fluidic connector 418 is sealingly engaged with an aperture of the neck 370 of the reservoir 404. The fluidic connector 418, which is shown apart in FIG. 22, enables a supply of oxygen to be provided through a first, longitudinal port of the connector 418 and into the inner volume of the reservoir 404 via a third, input port 464 while oxygen can be drawn from the inner volume of the reservoir 404 through a second, longitudinal port of the connector 418, which is in fluidic communication with ambient pressure tubing 422 and, through that tubing 422, the recipient as previously shown and described. Also as previously shown and described, the fluidic communication from the source to the connector 418 could, for instance, be through high-pressure tubing. While a single aperture in the neck 470 of the reservoir 404 is depicted in the present embodiment, it would be possible and within the scope of the invention to have separate, concentric, or otherwise disposed inflow and outflow apertures.

The decoupling reservoir 404 is again expandable and collapsible such that a volume of oxygen can be made available at ambient pressure for natural inhalation by a patient. In the present embodiment, however, the reservoir 404 is formed from a flexible shell portion 404A joined with a rigid shell portion 404B. Together, the flexible and rigid shell portions 404A and 404B form an oblong, generally egg-shaped decoupling reservoir 404 with oval horizontal and vertical cross sections while the housing 402 is again generally cube shaped. Within the scope of the invention, the flexible shell portion 404A could form just a portion of the overall shape of the reservoir 404 and be joined with the rigid shell portion 404B in a fluidically sealed manner. Alternatively and also within the scope of the invention, the flexible shell portion 404A could span the entire shape of the decoupling reservoir 404 and have some or all of its overlapping shape fixed to, such as by being nested and retained within, the rigid shell portion 404B. The rigid shell portion 404B defines an oblong, concave bowl that spans approximately one-half of the shape of the reservoir 404. The non-overlapping portion of the flexible shell portion 404A forms the other approximately half of the shape of the reservoir 404. The rigid shell portion 404B forms a lower half of the oblong, general egg shape of the reservoir 404, and the flexible shell portion 404A forms an oblong, concave bowl shape and an upper half of the overall oblong, general egg shape of the reservoir 404 when the reservoir 404 is inflated. The shell portions 404A and 404B could be readily reversed or otherwise disposed. The neck aperture 470 of the reservoir 404 is positioned to permit the reservoir 404, in this case the flexible shell portion 404A thereof, to expand and collapse smoothly and without risk of obstruction. In the depicted embodiment, the neck aperture 470 of the reservoir 404 is centered vertically and horizontally along a center longitudinal of the reservoir 404. However, it will be understood that the neck aperture 470 of the reservoir 404 could be otherwise positioned within the scope of the invention except as may be expressly limited by the claims.

The rigid shell portion 404B is fixed in position within the housing 402 such that the flexible shell portion 404A is derivatively fixed in position within the housing 402. The flexible shell portion 404A will thus rise away from the rigid shell portion 404B during inflation, and the flexible shell portion 404A will be drawn toward and potentially into the rigid shell portion 404B during deflation. In the present embodiment, the flexible and rigid shell portions 404A and 404B are retained within the housing 402 with the rigid shell portion 404B retained atop the bottom wall thereof, but it would also be possible for the rigid shell portion 404B itself to form an exterior portion of a housing.

With the position of the flexible shell portion 404A so fixed in relation to the housing 402, the volume and state of inflation of the reservoir 404 can be determined based on a sensing of, for example, the position within the housing 402 of the upper wall portion of the reservoir 404 as formed by the flexible shell portion 404A, whether by a contactless distance sensor, a contact switch, or another inflation detection system. In the present embodiment, plural distance sensors 456 are retained by the upper wall of the housing 402 facing the reservoir 404. The system 400 is thus capable of determining the present position and, with multiple sensors 456, the contour of the upper portion of the wall of the reservoir 404 formed by the flexible shell portion 404A. Based thereon, the system 400 is capable of automatically electronically calculating the present volume and state of inflation of the reservoir 404. Based on that determination of the present volume and state of inflation, replenishing gas can be automatically permitted to flow into the reservoir 404 or prevented from so flowing.

Together, the flexible shell portion 404A and the rigid shell portion 404B define an inner volume of the reservoir 404 that is sealed but for an entry and exit orifice in the neck 470 of the reservoir 404, although it would be within the scope of the invention to have separate entry and exit orifices. The rigid shell portion 404B can be formed from any rigid or semi-rigid material, and the rigid shell portion 404B can be continuous or discontinuous, such as where the flexible shell portion 404A spans the entire shape of the reservoir 404, provided that a continuous, substantially gas impermeable reservoir 404 is defined.

The decoupling reservoir 404 thus again comprises a shell comprising flexible material in the form of the flexible shell portion 404A that acts as an envelope for retaining a volume of oxygen at ambient pressure throughout the entire breathing cycle waiting to be naturally inhaled by the patient. Based on the structural properties of the flexible shell portion 404A as taught herein, including one or more of the material, material thickness, and structural configuration of the flexible shell portion 404A, and the performance deriving therefrom, the flexible shell portion 404A of the decoupling reservoir 404 and the decoupling reservoir 404 itself provide no or substantially no resistance to inflation when receiving oxygen from a source 406. Moreover, the decoupling reservoir 404 provides no or substantially no resistance to deflation when oxygen is inhaled therefrom by a patient.

When maintained at ambient pressure and without pressurization as taught herein, the material of the flexible shell portion 404A of the decoupling reservoir 404 and the decoupling reservoir 404 itself are incapable of acquiring or exhibiting elastic potential energy. As such, within the bounds of ambient pressure, a decoupling reservoir 404 that is deflated will not tend to resiliently spring and expand toward an expanded configuration, and a decoupling reservoir 404 that is inflated will not tend to resiliently spring and contract toward a retracted or collapsed configuration.

The flexible shell portion 404A of the reservoir 404 could, for example, be formed from a film of flexible polymeric material with or without a lining layer. The material defining the flexible shell portion 404A could, for example, comprise a film, alternatively referred to as a foil, formed by one or more layers of polymeric material with or without a metallic lining, such as a film of aluminum. Thus, one non-limiting material for the flexible shell portion 404A of the decoupling reservoir 404 that is incapable of acquiring or exhibiting elastic potential energy at ambient pressure is a lightweight, flexible film or foil. One such flexible foil is founded on a film of polymeric material, such as a film of polyester, even more particularly a film of biaxially oriented polyethylene terephthalate (BOPET). As used herein, the term “film” shall mean an exceedingly thin layer. To achieve the desired properties of being incapable of acquiring or exhibiting elastic potential energy at ambient pressure while demonstrating sufficient gas impermeability, the film of polymeric material comprising polyester and even more particularly biaxially oriented polyethylene terephthalate preferably has a thickness of less than 50 microns (0.002 inches). Even more preferably, such a film of polymeric material will have a thickness of less than 25 microns (0.001 inches). Ideally, the thickness of such a film of polymeric material has a thickness between 2 and 20 microns (between 0.0000787 inches and 0.000787 inches) with 18 microns (0.000709 inches) presently being most preferable and employed in embodiments of the system 400.

While biaxially oriented polyethylene terephthalate polyester film may be preferable in many circumstances for its tensile strength, toughness, and durability, another possible polymeric film operative as the wall material forming the flexible shell portion 404A of the decoupling reservoir 404 is low density polyethylene. The low density polyethylene film will again be sufficiently thin as to be incapable of acquiring or exhibiting elastic potential energy at ambient pressure while demonstrating sufficient gas impermeability. A low density polyethylene film with a thickness of less than 100 microns (0.004 inches) is preferred. More preferably, such a film of low density polyethylene film material will have a thickness of less than 25 microns (0.001 inches). Ideally, the thickness of such a film of polymeric material has a thickness between 5 and 20 microns (between 0.000197 inches and 0.000787 inches). While low density polyethylene and biaxially oriented polyethylene terephthalate polyester polymeric films are preferred, it will be understood that other polymeric films could be employed in practices of the invention except as may be expressly excluded by the claims.

The film of polymeric material forming the flexible shell portion 404A of the decoupling reservoir 404 can be metalized, such as with an aluminum film lining, thereby further reducing the permeability of the film. In one non-limiting practice, a film of aluminum or potentially another metal can be evaporated onto the film of polymeric material. For avoidance of doubt, the term “foil” as used herein shall not be interpreted to require metallization except as expressly set forth.

As is enabled by formation of the flexible shell portion 404A of the reservoir 404 of a lightweight, flexible polymeric film or other material incapable of acquiring or exhibiting elastic potential energy, the reservoir 404 once expanded tends even when it is at ambient pressure to substantially maintain an expanded shape and configuration, whether by its own structural integrity or otherwise, even when it is open to external ambient pressure, such as by a fluidic connection to the recipient 426 through ambient pressure tubing 422 connected to the orifice defined by the neck 470 of the reservoir 404. As taught herein, when expanded, the reservoir 404 in preferred embodiments does not significantly collapse on its own due to the weight of its walls. When filled with oxygen, the reservoir 404 thus temporarily stores a compartmented volume of oxygen at ambient pressure waiting to be drawn therefrom by the recipient 426 without spontaneously collapsing and exhausting oxygen therefrom. Concomitantly, again by virtue of being formed from a lightweight, flexible film or other material incapable of acquiring or exhibiting elastic potential energy at ambient pressure, the decoupling reservoir 404 provides substantially no resistance to collapse as the oxygen therein is naturally inhaled by the recipient 426. A patient can thus naturally inhale without opposition from the decoupling reservoir 404 as would exist, for instance, with a reservoir exhibiting elastic potential energy as would be the case with, for example, a resilient rubber bladder exhibiting elastic potential energy.

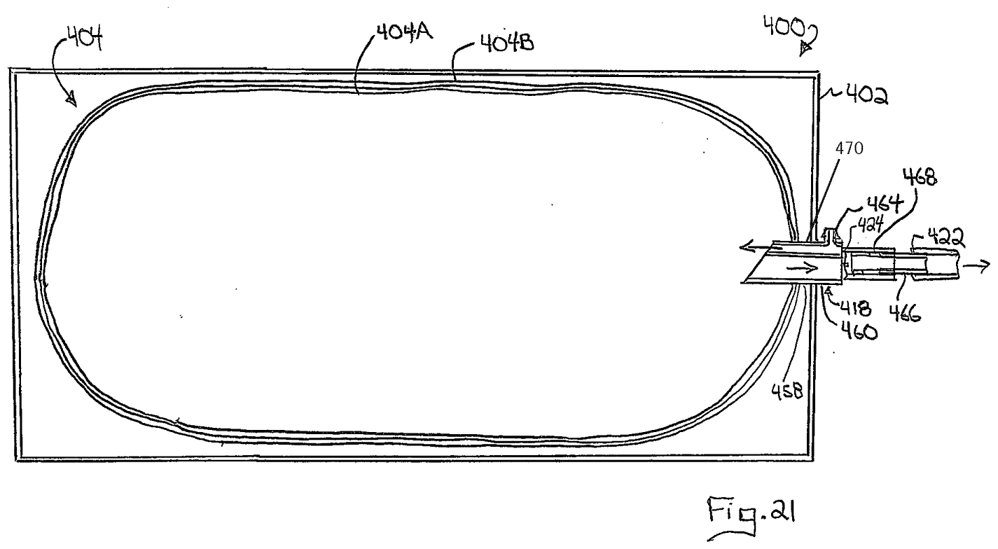

A fluidic connector 418 again has a first, longitudinal port in fluidic communication with the reservoir 404, such as through the aperture in the neck 470 of the reservoir 404. The fluidic connector 418 has a second, longitudinal port in fluidic communication with the ambient pressure tubing 422 and, through that tubing 422, the recipient. Finally, the fluidic connector 418 has a third, lateral port 464, which can be referred to as an injection port 464, between the first and second ports in fluidic communication with the oxygen source. Also as previously shown and described, the fluidic communication from the source to the connector 418 could, for instance, be through high-pressure tubing acting as a supply conduit from the oxygen source to an oxygen connector fixed to the housing 402 and high-pressure tubing from the oxygen connector to a supply valve. At least the first and second ports are in fluidic communication with one another within and through the fluidic connector 418. As shown in FIGS. 21 and 22, a channel is established within the connector 418 to direct oxygen received into the injection port 464 through the first, longitudinal port and into the inner volume of the reservoir 404.

As in FIG. 21, a one-way inspiratory valve 424 is interposed between the reservoir 404 and the recipient. In the depicted embodiment, the one-way inspiratory valve 424 is disposed distal to the injection port 464 with respect to the reservoir 404 but proximal to an air-input orifice 462 that permits ambient air to be drawn into the connector 418 to be entrained with oxygen drawn from the reservoir 404. The one-way inspiratory valve 424 is operative to allow gas to be drawn from the reservoir 404 through the first port and to be provided to a recipient through the second port while preventing gas from being passed from the second port and into the reservoir 404 through the first port, such as through the ambient-pressure tubing 422 during exhalation.

The connector 418 can incorporate further characteristics from the present inventors' application Ser. No. 18/371,127 for a Gas Blending Apparatus and Gas Delivery System with such a Gas Blending Apparatus, filed Sep. 21, 2023, the entirety of which being incorporated herein by reference. The connector 418, which may alternatively be referred to as a gas blending apparatus 418, can be seen to be founded on a tubular main conduit body 460 with a tubular neck portion 458, which may be integral or separable therefrom, that is sealingly received in the neck 470 of the reservoir 404.

The air-input orifice 462 permits the entrance of ambient air into the connector 418 while preventing that air from entering the reservoir 404. When the air-input orifice 462 is open, air will be drawn through the air-input orifice 462 to be blended and entrained with gas drawn from the reservoir 404. The ratio of ambient air drawn through the air-input orifice 462 to gas drawn from the reservoir 404 will be dependent on factors including the effective size of the aperture through the air-input orifice 462 and the effective size of that aperture in relation to the aperture bounded by the neck connector in the reservoir 404.

The effective size of the aperture provided by the air-input orifice 462 is selectively adjustable thereby to permit an adjustment of the ratio of ambient air drawn through the air-input orifice 462 and blended with gas drawn from the reservoir 404. One of ordinary skill in the art would be aware of plural mechanisms for selectively adjusting the effective size of the aperture provided by the air-input orifice 462 after being aware of the present disclosure. In the embodiment of the gas blending apparatus 418 depicted, adjustment of the effective size of the aperture provided by the air-input orifice 462 is provided by a tubular rotary adjustment member 466 that is rotatably engaged with the main conduit body of the gas blending apparatus 418. In the depicted embodiment, the ambient pressure tubing 422 is sealingly engaged with the tubular rotary adjustment member 466, and the rotary adjustment member 466 is rotatably engaged with the main conduit body of the gas blending apparatus 418. The rotary adjustment member 466 has a cylindrical proximal portion that is matingly received into the main conduit body 460 to overlap with the air-input orifice 462 and a cylindrical distal portion for being matingly engaged with the ambient-pressure tubing 422. The cylindrical proximal portion of the rotary adjustment member 466 has a plurality of differently sized apertures 468 circumferentially spaced therearound. While different permutations are certainly possible, the apertures 468 in the proximal portion of the rotary adjustment member 466 in the depicted example sequentially vary in size from an aperture 468 of a maximum size to an aperture 468 of a minimum size with a portion of the rotary adjustment member 466 having no aperture at all.

The apertures 468 are disposed within the rotary adjustment member 466 to align longitudinally with the air-input orifice 462. Accordingly, by a selective rotation of the rotary adjustment member 466 in relation to the main conduit body 460, a selected aperture 468 can be circumferentially aligned with the air-input orifice 462. Thus, when the largest aperture 468 of the rotary adjustment member 466 is selectively aligned with the air-input orifice 462, a maximum or greatest proportion of air will be drawn in through the orifice 462 to blend with the gas drawn from the reservoir 404. When the smallest aperture 468 is selectively aligned with the air-input orifice 462, the proportion of air drawn through the orifice 462 to blend with the gas drawn from the reservoir 404 will be at its smallest, and intermediate apertures 468 permit proportions of air drawn through the orifice 462 between the smallest and greatest proportions. The circumferential location of the rotary adjustment member 466 without an aperture 468 can be selectively aligned with the air-input orifice 462 to prevent ambient air from being drawn in through the orifice 462 and blended with the gas drawn from the reservoir 404 such that no air will be blended with the gas drawn from the reservoir 404. As in the '127 application, the gas blending apparatus 10 can have plural FiO2-setting, positive mechanical engagement formations on the rotary adjustment member 466 and the main conduit body 460 to permit the rotary adjustment member 466 to be disposed and retained in a known rotational orientation relative to the main conduit body 460. The rotary adjustment member 466 can thus be selectively positioned rotationally relative to the main conduit body 460 to provide direct and immediate control over the size of the aperture 468 that is aligned with the orifice 462 and thus to provide directly and immediate control over the fraction of inspired oxygen (Fi02) or other gas or gases delivered to the recipient.

The system 400 using such a gas blending apparatus 418 enables the achievement of a “shadow effect” wherein oxygen or another gas is provided at a desired saturation on demand with every breath without regard to respiratory frequency, volume, or other factors. The system 400 adjusts to a patient's breathing pattern immediately and automatically, including during periods of high stress, exercise, altitude change, and illness without significant alteration of oxygen concentration. By setting the effective size of the opening provided for drawing air into the gas blending apparatus 418, a consistent fraction of inspired oxygen (Fi02) or other gas or gases is provided to the recipient without a need for complex mechanical or software systems. Consequently, a recipient is able to maintain a desired oxygen-blood saturation (SaO2) even during changes in respiratory frequency and volume. Since the dispensing and conservation system 400 is passive with respect to the ventilation received by the patient, the patient's own breathing effectively controls the amount of oxygen provided at all times. Without requiring complex mechanisms or software and without repeated adjustments, the patient will automatically breathe in approximately the same fraction of inspired gases without regard to the volume or frequency of inspiration.

The volume of oxygen in the decoupling reservoir 404 is again retained substantially at ambient pressure and is not inflated to a state of pressurization. As a recipient inspires, oxygen will be drawn from the decoupling reservoir 404 through the ambient pressure tubing 422 thereby drawing from and tending to reduce the volume of oxygen in the decoupling reservoir 404. Due to the compressible nature of the decoupling reservoir 404 without elastic potential energy in the wall or walls forming the flexible shell portion 404A thereof, the flexible shell portion 404A and thus the reservoir 404 in general will tend to contract without imparting resistance to the natural inhalation of the patient. When the flexible shell portion 404A contracts by a sufficient amount, the reservoir 404 is automatically replenished with oxygen or other gas by operation of the inflation detection system without pressurization of the reservoir 404.

One skilled in the art may appreciate plural mechanisms that would operate as inflation detection systems to detect the state of inflation of the decoupling reservoir 404. In the present embodiment as referenced above, the inflation detection system is founded on plural distance sensors 456 retained in a fixed position facing the flexible shell portion 404A, in this case by the upper wall of the housing 402. The inflation detection system is thus capable of providing a continuous, real-time determination of the present position and, with multiple sensors 456, the contour of the upper portion of the wall of the reservoir 404 formed by the flexible shell portion 404A. Based thereon, the present volume and state of inflation of the reservoir 404 is automatically electronically calculated by the system 400.

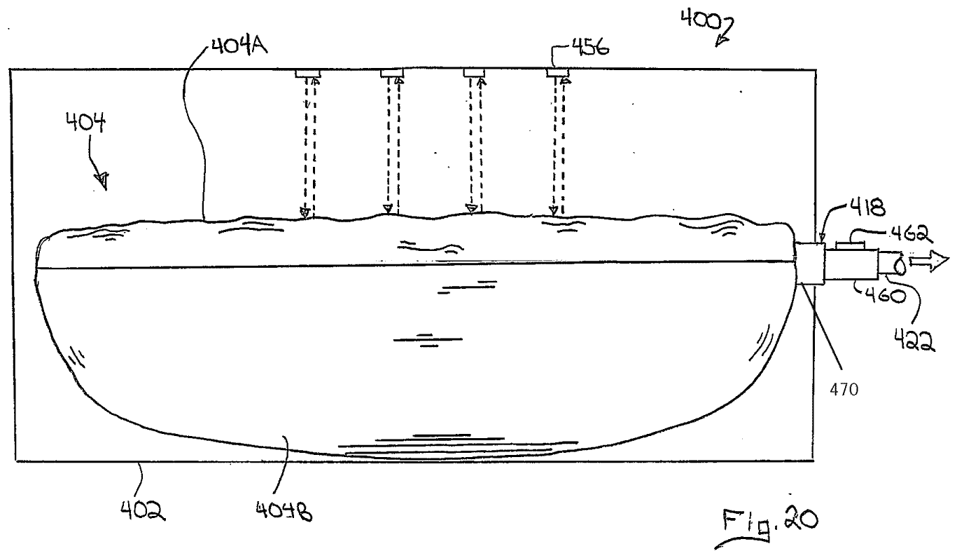

The inflation detection system operates as or as a component of or to actuate a flow switch as previously illustrated and discussed. When the distance sensors 456 detect based on the distance of the flexible shell portion 404A from the sensors 456 that the flexible shell portion 404A has reached a predetermined maximum state of inflation, such as is depicted in FIG. 19, and prior to pressurization, the supply valve is actuated to an OFF condition where gas is prevented from flowing from the source to the reservoir 404. Conversely, when the distance sensors 456 detect based on the increased distance of the flexible shell portion 404A from the sensors 456 that the flexible shell portion 404A has reached a predetermined minimum state of inflation, such as is depicted in FIG. 20, the distance sensors 456 cause an electronic signal to be sent to actuate the supply valve to an ON condition under which gas is allowed to flow from the source to the reservoir 404 to inflate the reservoir 404. Thus, the system 400 has a predetermined maximum distance or a combination of maximum distances as sensed by the sensors 456 that establish a threshold for triggering an electronic or other signal to actuate the supply valve to permit a replenishing flow of oxygen or other gas from the source. The decoupling reservoir 404 can thus be automatically inflated, such as to or within a given range of the maximum volume of the reservoir 404, without over-inflation or pressurization of the reservoir 404 to provide an available source of oxygen simply to be inhaled naturally by a patient in independent processes of inflation and inhalation. By relying on electronically sensed distances, reliability and performance are improved, and the risks of over filling and under filling are minimized.

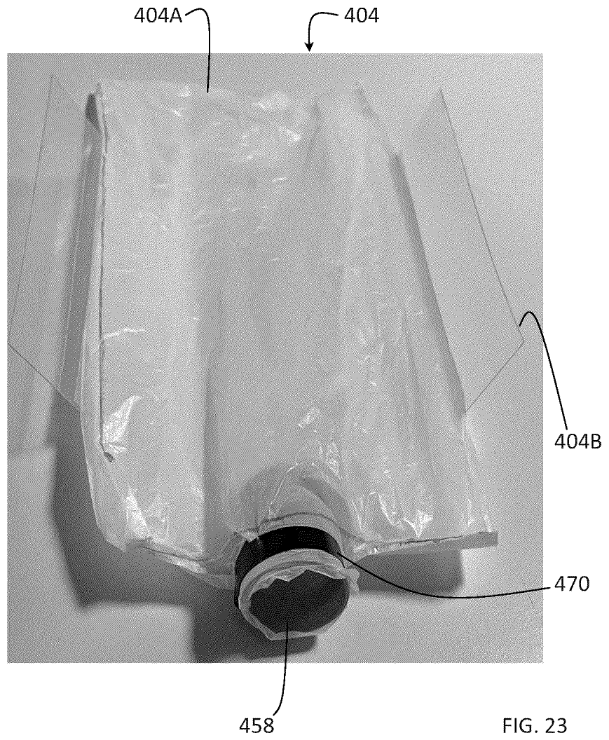

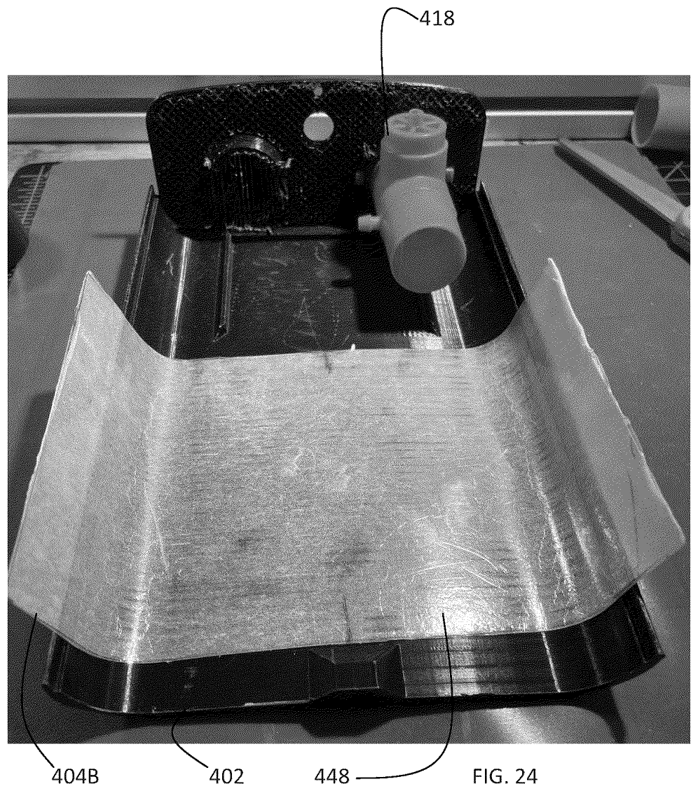

A further embodiment of the decoupling reservoir 404 is depicted in FIG. 23. There, the decoupling reservoir 404 again has a flexible shell portion 404A and a rigid shell portion 404B. Here, however, the flexible shell portion 404A when expanded defines an entire, enclosed envelope, which in this embodiment is generally cube-shaped, of the decoupling reservoir 404. The flexible shell portion 404A has a neck aperture 470 for allowing gas to enter and exit the inner volume of the reservoir 404, and a rigid neck sleeve 458 of the connector (not shown in FIG. 23) is matingly received into the neck aperture 470 of the reservoir 404 with a fluid-tight engagement established therebetween. The rigid shell portion 404B is semi-rigid and defines a U-shaped cradle that receives and supports an overlapping portion of the flexible shell portion 404A. As shown in FIG. 24, a layer of adhesive 448 is disposed over the facing surface of the rigid shell portion 404B. The overlapping portion of the flexible shell portion 404A is fixed to the rigid shell portion 404B by the layer of adhesive 448. Under this construction, the flexible shell portion 404A is able to expand and contract within and atop the U-shaped rigid shell portion 404B in an unimpeded manner to allow gas, such as oxygen, to be naturally inhaled therefrom by a patient and to be replenished therein from a source.

While a compressed gas tank is often depicted and referred to herein as the oxygen source 406 herein, other oxygen sources 406 are possible within the scope of the invention. By way of further, non-limiting examples, the automatic gas conservation system 400 can provide on-demand oxygen to patients with oxygen supplied by an oxygen concentrator. An oxygen concentrator does not require a tank. Instead, it takes in air and removes the nitrogen from it thereby leaving the oxygen-enriched gas for those patients requiring medical oxygen. The typical flow of this compressed oxygen is 1-5 liters/minute. High-end oxygen concentrators can deliver upwards of 50 L/minute, but they require more electricity and more maintenance.

By placing an automatic gas conservation system 400 as disclosed herein between the oxygen concentrator and the recipient 426, such as a patient face mask or a nasal cannula, an excess of oxygen can be stored at ambient pressure for use if, due to flow limitations, volume demands, or otherwise, sufficient supply is not provided by the concentrator. For example, if the oxygen concentrator is providing 10 L/minute and the patient suddenly needs more as his saturation level is dropping, there will be a volume of oxygen at ambient pressure available in the donor reservoir 404. Without the reservoir 404, the patient would be limited to the flow of the concentrator, which itself is limited. Therefore, without the reservoir 404, if a patient needs more oxygen to survive, the choices are to increase oxygen flow to the mask, which may be impossible, or intubate the patient and use mechanical ventilation, something which both doctor and patient want to avoid.

Where the oxygen source 406 is an oxygen concentrator, the automatic gas conservation system 400 can be placed between the oxygen concentrator and the patient mask 426 or other recipient so that, as oxygen leaves the concentrator, it enters the large reservoir 404 where it remains at ambient pressure until the patient inhales. As the patient breathes in and draws oxygen from the reservoir 404, the reservoir 404 begins to deplete, the supply valve 412 from the oxygen concentrator as the oxygen source 406 opens to replenish the reservoir 404 with compressed oxygen from the oxygen concentrator. As the patient exhales, no flow occurs between the reservoir 404 and the patient through the recipient mask 426 or otherwise. Rather than wasting the oxygen flowing from the concentrator during the exhalation phase of the patient, the flow is employed to replenish the reservoir 404. Once the reservoir 404 is filled to the predetermined maximum state of inflation, the supply valve 412 stops the flow of oxygen from the oxygen concentrator source 406. When the donor reservoir 404 contracts to below the predetermined minimum state of inflation, the shut off valve 412 opens to replenish the reservoir 404 with oxygen from the oxygen concentrator, and the cycle repeats. In this manner, oxygen not taken in by the patient during inspiration is stored rather than lost. In one example, a concentrator 406 with an output of 20 L/minute used with a patient needing only about 5 liters of highly concentrated oxygen during inhalation leaves 10 liters or more that could extend supply availability. Oxygen concentrators can thus be used for their intended purpose while having fewer demands with respect to work hours, electricity, wear and tear, and repairs thereby representing a more useful and reliable investment for the end user. Also, the system 400 and the concentrator as the oxygen source 406 cooperate to provide more reliable concentrations of oxygen to patients that require higher concentrations.

As disclosed herein, the automatic gas conservation system 400 and method make a gas, or a mixture of gases, available for inhalation from the donor reservoir 404 to the recipient 426 at ambient pressure. The gas or a mixture of gases at ambient pressure within the reservoir 404 can be drawn from the donor reservoir 404 when the recipient 426 drops its pressure below that of the donor reservoir 404, and the drawing of ambient pressure gas from the reservoir 404 stops immediately once the pressure of the recipient 426 equilibrates with that of the reservoir 404. The system 400 can provide a gas or a mixture of gases from the donor reservoir 404 to the recipient 426 at ambient pressure, and the percentage of gases in the mix reaching the recipient 426 can be regulated, such as by the resistance placed in the conduit of each gas involved in the mixture at ambient pressure.

The system 400 conserves gas from one or more sources 406 by limiting the flow of a continuous pressurized gas or gases to only when a recipient 426 creates the need for the gas or gases by dropping its pressure below the ambient pressure of the donor reservoir 404 and causing the reservoir 404 to fall below the predetermined minimum state of inflation. The donor reservoir 404 is thus capable of passively permitting the transfer of a gas or gases from an ambient pressure reservoir 404 by making the gas or gases available to the recipient 426 in a manner that matches the exact volume and speed of the demand based on the control of the pressure difference by the recipient 426. In embodiments of the system 400, the donor reservoir 404 is not only at ambient pressure but it is also large enough to accommodate the transfer in a completely passive way and without resistance of the volume of gas or gases in a 1:1 ratio at every point in time during the transfer from the beginning to the end of the flow created by the pressure difference between the recipient 426 and the donor reservoir 404, such as an inhalation phase during the respiratory cycle. In practices of the system 400, diagrams of the speed, pressure, time, and volume of patient inspiration and gaseous transfer from the donor reservoir 404 are equivalent and will likely be substantial mirror images. The drop in pressure of the recipient 426, such as during inhalation, is entirely used for the transfer of volume from the reservoir 404. No extra pressure or pressure differential is required to open a pressure check valve to start the flow as would be the case with a chamber or reservoir containing oxygen at a higher pressure than ambient pressure. The system 400 can work as a closed system, or it can be open to ambient pressure of the environment while being maintained at ambient pressure. The system 400 conserves gas or gases by limiting the flow of the gas or gases to the recipient 426 to a time only when they are needed. Since a patient intakes supplemental oxygen flowing to their recipient mask 426 only during inspiration, a far reduced volume of oxygen is required, such as one-half to one-third, as compared to continuous flow systems.

In practices of the invention, the system 400 can be used as a source to provide variable oxygen concentrations for CPAP machines used in the treatment of sleep apnea and COPD. The system 400 can help conserve oxygen from the pressurized source 406 by, for example, connecting the system 400 to the air input of the CPAP machine. The system 400 can also be employed to provide a reservoir 404 at ambient pressure for oxygen concentrators so patients can inhale or inspire a more reliable concentration of oxygen at ambient pressure, especially when high flows are demanded to treat a patient with respiratory insufficiency. Moreover, the system 400 can help oxygen concentrators as sources 406 of oxygen to provide the same oxygen concentration of oxygen to a patient with less required flow of oxygen, decreased hours of operation, reduced electricity consumption, increased longevity to the machine, and fewer repairs and parts. Still further, with the gas conserved, oxygen concentrators that previously supplied just one patient could potentially be used for plural patients concomitantly depending on the required supply rates.

As used herein, references to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, for example, the term “or” should generally be understood to mean “and/or.” Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” and the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics should be understood to contemplate a range of deviations that would be appreciated by one of ordinary skill in the art to operate satisfactorily for a corresponding use, function, or purpose. The use of any and all examples or exemplary language, as in “such as” or the like, provided herein, is intended merely to better illuminate the embodiments and does not pose a limitation on the scope of the embodiments. No language in the specification should be construed as indicating any unclaimed element as essential to the practice of the embodiments. In the description, it is understood that terms such as “first,” “second,” “top,” “bottom,” “upper,” “lower,” and the like are words of convenience and are not to be construed as limiting terms.

With certain details and embodiments of the present invention for a system for the decoupled supply and conservation of oxygen and other substances disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the invention. Those claims shall be deemed to include equivalent constructions insofar as they do not depart from the spirit and scope of the invention. It must be further noted that a plurality of the following claims may express, or be interpreted to express, certain elements as means for performing a specific function, at times without the recital of structure or material. As the law demands, any such claims shall be construed to cover not only the corresponding structure and material expressly described in this specification but also all legally-cognizable equivalents thereof.

The invention claimed is:

1. A system for the decoupled supply and conservation of gas to a patient, the system comprising:
an expandable and collapsible decoupling reservoir with an outer wall defining a shell, an inner volume for retaining a volume of gas, and at least one orifice for allowing a passage of gas into and out of the inner volume, wherein the outer wall of the decoupling reservoir comprises a lightweight, flexible film;
a supply conduit adapted to receive gas from a source of gas, wherein the supply conduit has a first end for supplying gas to the decoupling reservoir and a second end for being fluidically connected to the source of gas;
an ambient pressure conduit adapted to supply gas along a fluid path from the decoupling reservoir to a recipient, wherein the ambient pressure conduit has a first end in fluidic communication with the decoupling reservoir for receiving gas from the decoupling reservoir and a second end for being fluidically connected to the recipient;
an inflation detection system operable to detect a first condition wherein the decoupling reservoir is inflated with gas to a predetermined maximum state of inflation and a second condition wherein the decoupling reservoir is deflated to a predetermined minimum state of inflation; and
a valve system for being disposed between the source of gas and the decoupling reservoir wherein the valve system is operative when in a closed condition to prevent gas from flowing from the source of gas and into the decoupling reservoir when the decoupling reservoir is in the first condition, wherein the valve system is operative in an open condition to permit gas to flow from the source of gas and into the decoupling reservoir when the decoupling reservoir is in the second condition, and wherein the valve system and the inflation detection system are operative to maintain the volume of gas in the decoupling reservoir substantially at ambient pressure.

2. The system for the decoupled supply and conservation of gas of claim 1, wherein the decoupling reservoir has a fully inflated condition and wherein the predetermined maximum state of inflation is less than the fully inflated condition.

3. The system for the decoupled supply and conservation of gas of claim 2, wherein the decoupling reservoir has a fully deflated condition and wherein the predetermined minimum state of inflation is less than the predetermined maximum state of inflation but greater than the fully deflated condition.

4. The system for the decoupled supply and conservation of gas of claim 1, further comprising a source of gas comprising a source of oxygen.

5. The system for the decoupled supply and conservation of gas of claim 1, wherein the valve system comprises a solenoid valve that is in electrical communication with the inflation detection system, wherein the solenoid valve is induced by the inflation detection system to a closed condition to prevent the flow of oxygen from the source of oxygen to the donor reservoir when the decoupling reservoir is in the first condition, and wherein the solenoid valve is induced by the inflation detection system to an open condition to permit the flow of oxygen from the source of oxygen to the decoupling reservoir when the decoupling reservoir is in the second condition.

6. The system for the decoupled supply and conservation of gas of claim 1, further comprising a recipient delivery device coupled to the second end of the ambient pressure conduit wherein the recipient delivery device comprises a breathing mask.

7. The system for the decoupled supply and conservation of gas of claim 1, further comprising a housing wherein the decoupling reservoir is disposed within the housing, wherein the inflation detection system comprises a distance sensor retained by the housing, wherein the distance sensor is operative to detect a distance of the outer wall of the decoupling reservoir from the distance sensor, and wherein the inflation detection system is operative to detect the first condition and the second condition based on the distance of the outer wall of the decoupling reservoir from the distance sensor.

8. The system for the decoupled supply and conservation of gas of claim 7, wherein the inflation detection system comprises plural distance sensors retained by the housing and wherein the inflation detection system is operative to detect a contour of the decoupling reservoir based on localized distances of the outer wall of the decoupling reservoir from respective distance sensors and, based on the contour of the decoupling reservoir and the localized distances of the decoupling reservoir from respective distance sensors, the state of inflation of the decoupling reservoir.

9. The system for the decoupled supply and conservation of gas of claim 1, further comprising a one-way inspiratory valve disposed along the fluid path from the decoupling reservoir to the recipient wherein the one-way inspiratory valve is operative to enable gas to flow from the decoupling reservoir, through the ambient pressure conduit, and to the recipient but to prevent reverse flow of gas.

10. The system for the decoupled supply and conservation of gas of claim 1, wherein the outer wall of the decoupling reservoir comprises a lightweight, flexible film of polymeric material.

11. The system for the decoupled supply and conservation of gas of claim 10, wherein the outer wall of the decoupling reservoir comprises a polyester film.

12. The system for the decoupled supply and conservation of gas of claim 11, wherein the polyester film comprising the outer wall of the decoupling reservoir is metalized.

13. The system for the decoupled supply and conservation of gas of claim 10, wherein the outer wall of the decoupling reservoir comprises a film of biaxially oriented polyethylene terephthalate.

14. The system for the decoupled supply and conservation of gas of claim 10, wherein the outer wall of the decoupling reservoir comprises a lightweight, flexible film of polyethylene.

15. The system for the decoupled supply and conservation of gas of claim 14, wherein the outer wall of the decoupling reservoir comprises a lightweight, flexible film of low density polyethylene.

16. The system for the decoupled supply and conservation of gas of claim 1, wherein the lightweight, flexible film comprising the outer wall of the decoupling reservoir has a thickness of less than 100 microns (0.004 inches).

17. The system for the decoupled supply and conservation of gas of claim 16, wherein the lightweight, flexible film comprising the outer wall of the decoupling reservoir has a thickness of less than 25 microns (0.001 inches).

18. The system for the decoupled supply and conservation of gas of claim 17, wherein the lightweight, flexible film comprising the outer wall of the decoupling reservoir comprises a film of biaxially oriented polyethylene terephthalate with a thickness between 2 and 20 microns (between 0.0000787 inches and 0.000787 inches).

19. The system for the decoupled supply and conservation of gas of claim 17, wherein the lightweight, flexible film comprising the outer wall of the decoupling reservoir comprises a film of low density polyethylene with a thickness between 5 and 20 microns (between 0.000197 inches and 0.000787 inches).

20. The system for the decoupled supply and conservation of gas of claim 1, further comprising a fluidic connector, wherein the fluidic connector has a first port in fluidic communication with the decoupling reservoir, a second port in fluidic communication with the ambient pressure conduit and, through the ambient pressure conduit, to the recipient, and a third port in fluidic communication with the supply conduit adapted to receive gas from the source of gas wherein the third port is disposed between the first and second ports and further comprising a one-way inspiratory valve interposed between the decoupling reservoir and the recipient, wherein the one-way inspiratory valve is operative to enable oxygen to flow from the decoupling reservoir, through the ambient pressure conduit, and to the recipient but to prevent reverse oxygen flow from the recipient and into the decoupling reservoir.

21. The system for the decoupled supply and conservation of gas of claim 1, wherein the lightweight, flexible film of the decoupling reservoir forms a concave bowl, wherein the decoupling reservoir further comprises a rigid shell portion that forms a concave bowl, and wherein the concave bowl defined by the lightweight, flexible film and the concave bowl formed by the rigid shell portion are joined to form the decoupling reservoir.

22. A system for the decoupled supply and conservation of gas to a patient, the system comprising:
an expandable and collapsible decoupling reservoir with an outer wall defining a shell, an inner volume for retaining a volume of gas, and at least one orifice for allowing a passage of gas into and out of the inner volume, wherein the outer wall of the decoupling reservoir comprises a material incapable of acquiring or exhibiting elastic potential energy when the decoupling reservoir is at ambient pressure whereby the decoupling reservoir when deflated will not tend to resiliently spring and expand toward an expanded configuration and whereby the decoupling reservoir when inflated will not tend to resiliently spring and contract toward a collapsed configuration;
a supply conduit adapted to receive gas from a source of gas, wherein the supply conduit has a first end for supplying gas to the decoupling reservoir and a second end for being fluidically connected to the source of gas;
an ambient pressure conduit adapted to supply gas along a fluid path from the decoupling reservoir to a recipient, wherein the ambient pressure conduit has a first end in fluidic communication with the decoupling reservoir for receiving gas from the decoupling reservoir and a second end for being fluidically connected to the recipient;
an inflation detection system operable to detect a first condition wherein the decoupling reservoir is inflated with gas to a predetermined maximum state of inflation and a second condition wherein the decoupling reservoir is deflated to a predetermined minimum state of inflation; and
a valve system for being disposed between the source of gas and the decoupling reservoir wherein the valve system is operative when in a closed condition to prevent gas from flowing from the source of gas and into the decoupling reservoir when the decoupling reservoir is in the first condition, wherein the valve system is operative in an open condition to permit gas to flow from the source of gas and into the decoupling reservoir when the decoupling reservoir is in the second condition, and wherein the valve system and the inflation detection system are operative to maintain the volume of gas in the decoupling reservoir substantially at ambient pressure.

23. The system for the decoupled supply and conservation of gas of claim 22, wherein the outer wall of the decoupling reservoir comprises a lightweight, flexible film.

24. The system for the decoupled supply and conservation of gas of claim 23, wherein the outer wall of the decoupling reservoir comprises a lightweight, flexible film of polymeric material.

25. The system for the decoupled supply and conservation of gas of claim 23, wherein the lightweight, flexible film comprising the outer wall of the decoupling reservoir has a thickness of less than 100 microns (0.004 inches).
